(19) United States
(12) Patent Application Publication
(10) Pub. No.: US 2001/0031475 A1
(43) Pub. Date: Oct. 18, 2001

(54) Self-encoded combinatorial synthesis of compound multiplets

(76) Inventors: Mark A. Gallop, Los Altos, CA (US); William J. Dower, Menlo Park, CA (US); Ron W. Barrett, Saratoga, CA (US)

Correspondence Address: Gerald F. Swiss, BURNS, DOANE, SWECKER & MATHIS, L.L.P., P.O. Box 1404, Alexandria, VA, 22313-1404, US

(21) Appl. No.: 09/791,990

(22) Filed: Feb. 22, 2001

Related U.S. Application Data
(60) Provisional application No. 60/184,377, filed on Feb. 23, 2000.

(51) Int. Cl. G01N 33/53; C07K 1/04

(52) U.S. Cl. 435/7.1; 530/334

(57) ABSTRACT

The present invention provides a variety of methods for synthesizing, encoding and decoding compounds in a combinatorial library. One step or cycle in the synthetic methods of the invention is a self-encoding step in which different pairs of components, each pair with a known and different molecular weight difference, are reacted with supports, whereby two compounds differing in molecular weight are formed on each support. The molecular weight difference between the two compounds on the support encodes for a particular component pair. Libraries of compounds formed according to the methods of the invention are also provided.

This application claims priority under 35 U.S.C. § 120 to U.S. Provisional Patent Application Ser. No. 60/184,377 filed on Feb. 23, 2000; the entire content of which is hereby incorporated by reference.

### BACKGROUND OF THE INVENTION

Combinatorial synthesis of chemical libraries by the split and pool strategy has been firmly established as an efficient method for generating large numbers of synthetic compounds for biological or chemical evaluation in just a few synthetic steps. The bead-based library format allows one to treat such compound collections either as complex mixtures (e.g., after cleaving the compounds from a pool of beads), or as individual compounds by manipulation and cleavage of products from single beads (since each bead contains, ideally, just one type of molecule).

A variety of techniques have been introduced to identify active members of such libraries, as recently reviewed in Gallop et al., (1994) J. Med. Chem., 37: 1233 and Balkenhohl et al., (1996) Angew. Chem. Int. Ed. Engl. 35: 2288. For screening soluble compound mixtures, the most commonly used methods rely on some form of deconvolution strategy in which a series of smaller sub-pools of compounds are prepared and assayed so as to fractionate the original mixture into its most active single component(s).

Extraction and identification of active components from soluble mixture libraries has been achieved by various affinity selection techniques followed by mass spectroscopic analysis (see, e.g., Chu, et al., (1996) .J Am. Chem. Soc. 118: 7827; Weiboldt, et al., (1997) Anal. Chem. 69: 1683). As the complexity of the library increases, simply detecting a molecular ion in the mass spectrum of the analyte often does not provide for unambiguous identification of the active component, as many library members may have the same molecular mass. In these cases, use of MS-MS fragmentation techniques can be helpful, though such methods typically require sophisticated instrumentation and are timeconsuming (see, e.g., Winger, et al., (1996) Rapid Commun. Mass Spectrom., 10: 1813; and Dunayevsky, et al., (1996) Proc. Natl. Acad. Sci. USA 93: 6157).

In cases where individual synthesis particles from combinatorial libraries are submitted to biological or other types of assay, the structure elucidation problem can be handled in a number of different ways. Because beads of diameter of greater than 100 μm typically used for solid phase synthesis contain hundreds of picomoles of compound, mass spectrometry is generally a sufficiently sensitive method to provide molecular weight information for any given library member (see, e.g., Egner, et al., (1995) J. Org. Chem. 60: 2652; Brummel et al., (1996) Anal. Chem. 68: 237). However, as previously noted, the mass redundancy inevitable with larger libraries leads to ambiguities that cannot be resolved on the basis of simple molecular ion information alone. For small libraries comprised of variable building blocks (monomers or components) selected from two different sets, a set of rules for choosing the building blocks such that every library member has a unique mass and can hence be readily identified by single bead MS has been defined (see, Hughes, (1998) J. Med. Chem. 41: 3804; and PCT Application WO 97/08190). However, this method is most useful for libraries that are not much larger than a few hundred members. For peptide libraries comprised of the conunon a-amino acids, conventional Edman sequencing methods has been applied with single beads to deduce the structure of the associated compounds (Lam, et al., (1991) Nature 354: 82). Youngquist, et al. (J. Am. Chem. Soc., 117: 3900 (1995)) have introduced a method for rapidly sequencing a peptide library member from a single resin bead by mass spectrometry, wherein a capping reagent is used at each synthetic step to effect partial termination of the growing polymer. The ladder of molecular ions observed in the mass spectrum of this synthetic product is used to reconstruct the sequence of addition of amino acid monomers. This method is limited to syntheses in which a partial termination can be readily achieved (e.g., oligomeric molecules) and also suffers from the fact that the terminated fragments are typically of low abundance and can be difficult to visualize by MS.

A variety of encoding strategies have been introduced that allow the reaction history of synthesis particles that have undergone split and pool synthesis to be deduced (see, e.g., Czarnik, (1997) Curr. Opin. Chem. Biol. 1: 60). Broadly speaking these methods can be categorized either as: (i) those in which identifier tags are added to or are modified on synthesis particles at each step of a multistep reaction sequence, or (ii) those in which identifier tags are previously associated with each synthesis particle, such that every particle has a uniquely distinguishable tag (barcode) that can be used to track the overall pathway experienced by the particle during the split and pool procedure.

Dower and coworkers have reported the use of methods in the first category for synthesizing libraries that employ various tags with distinguishable physical properties, including oligonucleotides, fluorophores and amines which can be used in binary or higher order combinations (see, e.g., Needels, et al., (1993) Proc. Natl. Acad. Sci. USA 90: 10700; Ni, et al., (1996) J. Med. Chem. 39: 1601; Dower, et al., U.S. Pat. No. 5,708,153, Dower, et al., U.S. Pat. No. 5,789,162; and Gallop, et al., U.S. Pat. No. 5,846,839). Similarly, Still et al. have discussed a method for identifying compounds from a library by reference to a set of identifiers which encode each of the reaction stages associated with the synthesis (Ohlmeyer, et al., (1993) Proc. Natl. Acad. Sci. USA 90: 10922; Still, et al, U.S. Pat. No. 5,565,324 and U.S. Pat. No. 5,789,172). Other groups have reported methods for identifying compounds produced through a series of one or more reactions by concurrent covalent attachment of specifically distinguishable fluorophore tags that are uniquely associated with each component in the synthesis (Egner, et al., (1997) J. Chem. Soc. Chem. Commun. 735; Scott, et al., (1997) Bioorg. Med. Chem. Lett. 7: 1567; Furka, et al., PCT Application WO 93/24517; and Seul, et al., PCT Application WO 98/53093). Trau has discussed the use of non-covalent forces to associate distinguishable small fluorescent reporter beads with larger synthesis particles to achieve a similar coding effect (Trau, et al., PCT Application WO 99/24458). Yet others have proposed coding methodologies based upon tags distinguishable by mass spectrometry (Geysen, et al., (1996) Chem. Biol. 3: 679), infra-red or Raman spectroscopy (Hochlowski, et al., PCT Application WO 98/11036) and ¹⁹F N.M.R. spectroscopy (Hochlowski, et al., (1999) J. Comb. Chem. 1: 291; and Hochlowski, et al., PCT Application WO 99/19344).

Coding methods from the second category have been reported by various groups and include the use of radiofrequency transponders encapsulated within packets of synthesis resin, which can be taken through a split and pool synthesis and scanned individually at each splitting step to record the reaction history of the resin (see, e.g., Moran, et al., (1995) J. Am. Chem. Soc. 117: 10787; Nicolaou, et al., (1995) Angew. Chem. Int. Ed. Engl. 34: 2289; Nova, et al., U.S. Pat. No. 5,741,462; and Nova, et al., U.S. Pat. No. 5,961,923). Other workers have discussed the use of composite synthesis particles equipped with optically distinguishable features readable by machine that allow the particle to be tracked at each step of the synthesis (see, e.g., Xiao, et al., (1997) Angew. Chem. Int. Ed. Engl. 36: 780; Kaye, et al., GB Application 2306484; Barrett, PCT Application WO 97/32892; Garman, et al., PCT Application WO 98/47838; and Corless et al., PCT Application WO 98/46550).

### SUMMARY

A variety of methods for synthesizing, encoding and decoding combinatorial libraries are disclosed herein, as are methods for screening such libraries to identify members that have an activity of interest. Libraries of compounds prepared using such methods are also provided.

The methods are based in part on an encoding strategy in which one step of the synthesis, referred to as a mixed coupling step or cycle, involves preparing a mixture or multiplet of compounds on each support. During this step, pairs of components are added to supports within each reaction vessel, instead of adding a single component to each vessel as is done in conventional combinatorial synthesis methods. Different pairs of components are added to different reaction vessels. These different pairs each have a known and distinctive difference in molecular weight, thereby providing a scheme that encodes for each pair of components. By adding pairs of components to a reaction vessel, multiplets or pairs of compounds are formed on each support. Because the molecular weight difference between the pair of components incorporated into these multiplets is known, one can determine the identity of a component in the compounds formed on a support from the difference in molecular weight of the compounds.

Thus, certain screening methods involve:

(a) conducting a plurality of synthesis cycles to synthesize compounds on supports in a component-by-component fashion, a synthesis cycle comprising apportioning supports into reaction vessels and reacting the supports in different vessels with different components of the compounds, whereby the components attach to the supports or with components attached to the supports in previous cycles, and the supports from different vessels are pooled between synthesis cycles;

wherein at least one cycle is conducted by contacting different vessels of supports with different paired components, the members of each pair of components attaching independently to the supports or components attached thereto in a previous cycle, whereby supports in the same vessel receive the same pair of components, and supports in different vessels receive different pairs of components, the components in each pair having a known difference in molecular weight, and the differences in molecular weights varying between pairs, to produce a population of supports bearing different pairs of compounds, the members of the pairs of compounds having a known difference in molecular weight;

(b) assaying the supports bearing different paired compounds, and isolating at least one support wherein at least one of the paired compounds on the isolated support has a desired property; and

(c) performing a determining step comprising determining the molecular weights of each of the compounds of the pair borne by the at least one isolated support, the difference in molecular weight between the members of a pair of compounds indicating which pair of components was incorporated into the pair of compounds in the at least one cycle.

The use of a mixed coupling step can be used in combination with other encoding strategies to provide multistep encoding schemes that enable one to determine each component of a compound that exhibits a desired activity. Certain methods utilize a preencoding scheme during the initial synthesis cycle. In this scheme, the supports in different reaction vessels are distinguishable from one another such that components added to different reaction vessels during this initial cycle become attached to different supports. Thus, the initial component of a compound can be determined from the identity of the support. The supports can be distinguished based upon a variety of different characteristics such as a physical characteristic or other label associated with the support.

Spatial encoding strategies can also be utilized with the mixed coupling encoding strategy to encode additional components. The spatial encoding strategy typically involves tracking the identity of the final components added into each of the different reaction vessels. Rather than pooling the final compounds formed in the different reaction vessels, compounds from different reaction vessels are separately assayed. In this way, one can determine the identity of the final component for a compound that has the desired activity based upon the location from which the compound was taken. Other methods utilize a plurality (typically two) of mixed coupling steps to encode multiple components.

Such combinations of encoding schemes can be used in a variety of methods involving 3, 4, 5 or more synthesis steps to prepare a library of compounds that can subsequently be screened for a desired activity. The activity screened for can include any number of activities including biological activities (e.g., capacity to bind a receptor, the capacity to be transported into or through a cell, the capacity to be a substrate or inhibitor for an enzyme, the capacity to kill bacteria, and/or the capacity to agonize or antagonize a receptor) or non-biological activities (e.g., a particular conductivity, resistivity, or dielectric property).

For example, certain screening methods involve a three-step synthesis utilizing mixed coupling and pre-encoding steps to encode for two components of the compound and involve:

(a) in a first synthesis cycle, apportioning a collection of labeled supports comprising different labels into a plurality of first reaction vessels so that the labeled supports in a reaction vessel are the same, but the labeled supports in different reaction vessels are different; and reacting the supports with different first components in the different first vessels, whereby the first components attach to the support either directly or optionally via some linker or spacer component;

(b) in a second synthesis cycle, pooling the supports, and apportioning the supports in a second plurality of reaction vessels, and reacting the supports with different paired components, the members of each pair having a known difference in molecular weight, the difference in molecular weight differing between pairs, whereby the members of each pair attach independently to the support via a component added in a preceding step;

(c) in a third synthesis cycle, pooling the supports and apportioning the supports in a third plurality of reaction vessels, and reacting supports with different third components in the different reaction vessels, whereby the third components attach to the support via a component added in a preceding step;

thereby forming a population of supports, each support bearing different pairs of compounds, the members of the pairs of compounds having a known difference in molecular weight;

(d) assaying the supports bearing different paired compounds, and isolating at least one support wherein at least one of the paired compounds on the isolated support has a desired property; and

(e) determining the molecular weights of each of the paired compounds borne by the at least one isolated support, the difference in molecular weight between the pair of compounds indicating which pair of components was incorporated into the pair of compounds in the second synthesis cycle, the labeling indicating which component was added during the first synthesis cycle, and the total molecular weight of each compound, and the identity of the components added during the first and second synthesis cycles, indicating which component was added during the third synthesis cycle.

Other three step combinatorial synthesis and screening methods utilize a combination of mixed coupling and spatial encoding and involve:

(a) in a first synthesis cycle, apportioning a plurality of supports into a plurality of first reaction vessels; and reacting the supports with different first components in the different vessels, whereby the first components attach to the support or to a component added in a previous step;

(b) in a second synthesis cycle, pooling the supports, and apportioning the supports into a plurality of second reaction vessels, and reacting the supports with different paired components, the members of each pair having a known difference in molecular weight, the difference in molecular weight differing between pairs, whereby the members of each pair attach independently to the support via a component added in a preceding step;

(c) in a third synthesis cycle, pooling the supports and apportioning the supports in a third plurality of reaction vessels, and reacting supports with different third components, whereby the third components attach to the support via a component added in a preceding step, and wherein the identity of each component in each reaction vessel is tracked such that the identity of the third component in each of the third reaction vessels is known;

thereby forming a population of supports, each support bearing different pairs of compounds, the members of the pairs of compounds having a known difference in molecular weight;

(d) separately assaying the supports bearing the paired compounds from each of the plurality of third reaction vessels, and isolating at least one support wherein at least one of the paired compounds on the isolated support has a desired property; and

(e) determining the molecular weights of each of the paired compounds borne by the at least one isolated support, the difference in molecular weight between the pair of compounds indicating which pair of components was incorporated into the pair of compounds in the second synthesis cycle, the identity of the third reaction vessel from which the support was obtained for the assaying step indicating which component was added during the third synthesis cycle, and the total molecular weight of each compound, and the identity of the components added during the second and third synthesis cycles, indicating which component was added during the first synthesis cycle.

A variety of four cycle combinatorial synthesis and screening methods are provided in which various combinations of pre-encoding, spatial encoding and one or two cycles of mixed coupling encoding strategies are utilized. In certain of these methods, one component is pre-encoded, another spatially encoded and yet another encoded in a mixed coupling step. Such methods involve:

(a) in a first synthesis cycle, apportioning a collection of labeled supports comprising different labels into a plurality of first reaction vessels so that the labeled supports in a reaction vessel are the same, but the labeled supports in different reaction vessels are different; and reacting the supports with different first components in the different first vessels, whereby the first components attach to the support;

(b) in a second synthesis cycle, pooling the supports, and apportioning the supports in a plurality of second reaction vessels, and reacting the supports with different paired components, the members of each pair having a known difference in molecular weight, the difference in molecular weight differing between pairs, whereby the members of each pair attach independently to the support via a component added in the preceding step;

(c) in a third synthesis cycle, pooling the supports and apportioning the supports in a plurality of third reaction vessels, and reacting the supports with different third components, whereby the third components attach to the support via a component added in a preceding step;

(d) in a fourth synthesis cycle, pooling the supports and apportioning the supports in a plurality of fourth reaction vessels, and reacting supports with different components, whereby the components attach to the support via a component added in the preceding step; and wherein the identity of each fourth component in each reaction vessel is tracked such that the identity of the fourth component added to each of the fourth reaction vessels is known;

thereby forming a population of supports, each support bearing different pairs of compounds, the members of the pairs of compounds having a known difference in molecular weight;

(e) separately assaying the supports bearing the paired compounds from each of the plurality of fourth reaction vessels, and isolating at least one support wherein at least one of the paired compounds on the isolated support has a desired property; and

(f) determining the molecular weights of each of the paired compounds borne by the at least one isolated support, the difference in molecular weight between the pair of compounds indicating which pair of components was incorporated into the pair of compounds in the second synthesis cycle, the labeling indicating which component was added during the first synthesis cycle, the identity of the reaction vessel from which the support was obtained for the assaying step indicating which component was added during the fourth synthesis cycle and the total molecular weight of each compound, and the identity of the components added during the first, second and fourth synthesis cycles, indicating which component was added during the third synthesis cycle.

In other synthesis and screening methods that include four different synthesis cycles, the components added during two cycles are encoded using mixed coupling and components during another cycle are spatially encoded. Certain of these methods involve:

(a) in a first synthesis cycle, apportioning a plurality of supports into a plurality of first reaction vessels, and reacting the supports with different first components in the different vessels, whereby the first components attach to the support or to a component added in a preceding step;

(b) in a second synthesis cycle, pooling said supports and apportioning the supports in a plurality of second reaction vessels, and reacting the supports with a first set of different paired components, the members of each pair having a known difference in molecular weight, the difference in molecular weight differing between pairs, whereby the members of each pair attach independently to the support or to the support via a component added in a preceding step;

(c) in a third synthesis cycle, pooling the supports and apportioning the supports in a plurality of third reaction vessels, and reacting the supports with a second set of different paired components, the members of each second pair having a known difference in molecular weight, the difference in molecular weight differing between the second pairs, whereby the members of each second pair attach independently to the support or to the support via a component added in a preceding step;

(d) in a fourth synthesis cycle, pooling the supports and apportioning the supports in a plurality of fourth reaction vessels, and reacting supports with different components, whereby the components attach to the support via a component added in a preceding step, and wherein the identity of each fourth component in each reaction vessel is tracked such that the identity of the fourth component added to each of the fourth reaction vessels is known;

thereby forming a population of supports, each support bearing four different compounds;

(e) separately assaying the supports bearing the four compounds from each of the fourth plurality of reaction vessels, and isolating at least one support wherein at least one of the four compounds on the isolated support has a desired property; and

(f) determining the molecular weights of the four compounds borne by the at least one isolated support, the difference in molecular weight between the members of a first pair of compounds from the four compounds indicating which pair of components was incorporated into the pair of compounds in the second synthesis cycle, the difference in molecular weight between the members of a second pair of compounds from the four compounds indicating which pair of components was incorporated into the pair of compounds in the third synthesis cycle, the location from which the support was obtained in the fourth synthesis cycle indicating which component was added during the fourth synthesis cycle, and the total molecular weight of each compound, and the identity of the components added during the second, third and fourth synthesis cycles, indicating which component was added during the first synthesis cycle.

Other methods involving four synthesis cycles are similar to the method just described, except that components in one cycle are pre-encoded rather than spatially encoded. Some of these methods involve:

(a) apportioning a plurality of supports into a plurality of first reaction vessels;

(b) in a in a first synthesis cycle, reacting the supports with different first components in the different vessels, whereby the first components attach to the support;

(c) reacting the supports with different labels in the different reaction vessels, such that supports within a reaction vessel bear the same label, but supports within different reaction vessels bear different labels;

(d) in a second synthesis cycle, pooling said supports and apportioning the supports in a plurality of second reaction vessels, and reacting the supports with a first set of different paired components, the members of each pair having a known difference in molecular weight, the difference in molecular weight differing between pairs, whereby the members of each pair attach independently to the support or to the support via a component added in a preceding step;

(e) in a third synthesis cycle, pooling the supports and apportioning the supports in a plurality of third reaction vessels, and reacting the supports with a second set of different paired components, the members of each second pair having a known difference in molecular weight, the difference in molecular weight differing between the second pairs, whereby the members of each second pair attach independently to the support or to the support via a component added in a preceding step;

(f) in a fourth synthesis cycle, pooling the supports and apportioning the supports in a plurality of fourth reaction vessels, and reacting the supports with different components, whereby the components attach to the support via a component added in a preceding step;

thereby forming a population of supports, each support bearing four different compounds;

(g) assaying the supports bearing the four compounds from each of the fourth plurality of reaction vessels, and isolating at least one support wherein at least one of the four compounds on the isolated support has a desired property; and

(h) determining the molecular weights of the four compounds borne by the at least one isolated support, the difference in molecular weight between the members of a first pair of compounds from the four compounds indicating which pair of components was incorporated into the pair of compounds in the second synthesis cycle, the difference in molecular weight between the members of a second pair of compounds from the four compounds indicating which pair of components was incorporated into the pair of compounds in the third synthesis cycle, the labeling indicating which component was added in the first synthesis cycle, and the total molecular weight of each compound, and the identity of the components added during the firs, second and third synthesis cycles, indicating which component was added during the fourth synthesis cycle.

Still other methods involve five synthesis rounds, and employ pre-encoding, spatial encoding and mixed coupling to encode for the components added during the cycles. Certain of these methods involve:

(a) in a first synthesis cycle, apportioning a plurality of supports into a plurality of first reaction vessels and reacting the supports with different first components in the different vessels, the first components attaching to the supports;

(b) in a second synthesis cycle,

(i) splitting the supports from each of the plurality of first reaction vessels into a set of multiple reaction vessels, the sets forming a plurality of second reaction vessels;

(ii) labeling the supports in each of the second reaction vessels with a different label, such that supports in a reaction vessel have the same label, but supports in different reaction vessels have different labels; and

(iii) reacting the supports in different reaction vessels of each set with different second components, whereby the second component attaches to the support via the first component;

(c) in a third synthesis cycle, pooling the supports from the plurality of second reaction vessels and reacting the supports with different third components in the different vessels, whereby the third components attach to the supports via the components added in a previous step;

(d) in a fourth synthesis cycle, pooling the supports, and apportioning the supports in a plurality of fourth reaction vessels; apportioning the supports in a plurality of third reaction vessels; and reacting the supports with different paired components, the members of each pair having a known difference in molecular weight, the difference in molecular weight differing between pairs, whereby the members of each pair attach independently to the support via a component added in a preceding step;

(e) in a fifth synthesis cycle, pooling the supports and apportioning the supports in a plurality of fifth reaction vessels, and reacting supports with different components, whereby the components attach to the support via a component added in the preceding step;

thereby forming a population of supports bearing different pairs of compounds, the members of the pairs of compounds having a known difference in molecular weight;

(f) separately assaying the supports bearing the paired compounds from each of the fifth plurality of reaction vessels, and isolating at least one support wherein at least one of the paired compounds on the isolated support has a desired property; and

(g) determining the molecular weights of each of the paired compounds borne by the at least one isolated support, the difference in molecular weight between the pair of compounds indicating which pair of components was incorporated into the pair of compounds in the fourth synthesis cycle, the labeling indicating which compounds were added in the first and second synthesis cycles, the fifth reaction vessel from which the support was obtained for the assaying step indicating which component was added during the fifth synthesis cycle, and the total molecular weight of each compound, and the identity of the components added during the first, second, fourth and fifth synthesis cycles, indicating which component was added during the third synthesis cycle.

Methods for synthesizing combinatorial libraries that incorporate the mixed coupling encoding strategy are also provided. For example, some methods involve: conducting a plurality of synthesis cycles to synthesize compounds on supports in a component-by-component fashion, a synthesis cycle comprising apportioning supports into reaction vessels and reacting the supports in different vessels with different components of the compounds, whereby the components attach to the supports or with components attached to the supports in previous steps, and the supports from different vessels are pooled between synthesis cycles;

wherein at least one cycle is conducted by contacting different vessels of supports with different first paired components, the members of each first pair attaching independently to the supports or components attached thereto in a previous cycle, whereby supports in the same vessel receive the same pair of components, and supports in different vessels receive different pairs of components, the components in each first pair having a known difference in molecular weight, and the differences in molecular weights varying between pairs, to produce a population of supports bearing different pairs of compounds, the members of the pairs of compounds having a known difference in molecular weight.

Libraries of compounds on supports are also provided. The members of such libraries each comprise a support and a first and second compound of differing composition attached to the support, wherein the first and second compounds (i) comprise n components joined to one another via chemical bonds, and (ii) differ from each other in molecular weight, the difference in molecular weight encoding for a component of the first and second compound, and wherein the nth component is the same for the first and second compound. In some instances, members of the library are labeled.

### BRIEF DESCRIPTION OF THE DRAWINGS

FIG. 1 illustrates a conventional split and pool synthesis including three chemical steps.

FIG. 2 depicts a self-encoded split and pool synthesis of compound pairs according to one example of the method of the invention involving three chemical steps.

FIG. 3 summarizes the steps of the synthesis of a 4000-member tripeptide library using orthogonal protecting group chemistry according to a method of the invention.

FIG. 4 summarizes the steps of the synthesis of a 4000-member tripeptide library using isokinetic monomer mixture coupling according to one method of the invention.

FIG. 5 depicts the synthesis of a 4096-member N-acyl-N-alkyl amino acid amide library according to one method of the invention.

FIG. 6 illustrates the building blocks for an N-acyl-N-alkyl amino acid library with fluorescent pre-encoding of amine components and mixture self-encoding of aldehyde components for a four-step coupling method of the invention.

FIG. 7 shows the synthesis of a 9216-member 1,5 benzodiazepin-2-one library synthesized according to a five-step coupling method of the invention.

FIG. 8 shows pairings of boronic acid building blocks for a 1,5-benzodiazepin-2-one library and molecular weight differences between the pairs which encode for a specific boronic acid pair.

FIG. 9 shows building blocks for a 9216-member 1,5-benzodiazepin-2-one library for use in a five-step coupling method of the invention.

FIG. 10 depicts a two-membrane system for assaying for transport through a cell.

### DETAILED DESCRIPTION OF THE SPECIFIC EMBODIMENTS

I. Definitions

The terms “polypeptide,” “protein” and “peptide” are used interchangeably and to refer to a polymer of amino acid residues. The term also applies to amino acid polymers in which one or more amino acids are chemical analogues of a corresponding naturally-occurring amino acid.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

A “ligand” refers to a molecule that is recognized by a particular receptor. The term does not imply any particular size or type of molecule. Thus, the term ligand includes, but is not limited to, a polypeptide, an oligosaccharide, a sugar, a hormone, an enzyme substrate, inhibitor or cofactor and a drug. A ligand can be the natural ligand of a receptor or a functional analogue thereof that can act, for example, as an antagonist or agonist.

A “receptor” is a molecule that has an affinity for a particular ligand. Receptors can be naturally-occurring or prepared using synthetic methods. Receptors can be used in the unaltered or natural state or aggregated with other receptors or species. Receptors that can be utilized in the screening methods of the invention include, but are not limited to, cell-surface receptors, antibodies, lectins, transport proteins, enzymes, cellular membranes and organelles, antisera reactive with particular antigenic determinants. Receptors include those proteins capable of transducing a signal across a cell membrane, including, for example, hormone receptors, ion channels (e.g., calcium, sodium or potassium channels), growth factor receptors, ligand-gated ion channels (e.g., acetyl choline receptors), adrenergic receptors, dopamine receptors and adhesion proteins (e.g., integrins and selectins).

A “transport protein” is a protein that has a direct or indirect role in transporting a molecule into, and/or out of and/or through a cell. The term includes, for example, membrane-bound proteins that recognize a substrate and effect its entry into a cell by a carrier-mediated transporter or by receptor-mediated transport. A transport protein is sometimes referred to as a transporter protein. The term also includes intracellularly expressed proteins that participate in trafficking of substrates through or out of a cell. The term also includes proteins or glycoproteins exposed on the surface of a cell that do not directly transport a substrate but bind to the substrate holding it in proximity to a receptor or transporter protein that effects entry of the substrate into or through the cell. Examples of carrier-mediated transporter include: the intestinal and liver bile acid transporters, dipeptide transporters, oligopeptide transporters, simple sugar transporters (e.g., SGLT1), phosphate transporters, monocarboxcylic acid transporters, P-glycoprotein transporters, organic anion transporters (OATP), and organic cation transporters. Examples of receptor-mediated transport proteins include: viral receptors, immunoglobulin receptors, bacterial toxin receptors, plant lectin receptors, bacterial adhesion receptors, vitamin transporters and cytokine growth factor receptors. A “substrate” of a transport protein is a compound whose uptake into or passage through a cell is facilitated by the transport protein. When used in relation to a transport protein, the term “ligand” includes substrates and other compounds that bind to the transport protein without being taken up or transported through a cell. Some ligands by binding to the transport protein inhibit or antagonize uptake of the compound or passage of the compound through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “naturally occurring” as applied to an object refers to the fact that an object can be found in nature.

II. Overview

The present invention provides a variety of methods for synthesizing, encoding and decoding compounds in a combinatorial library. The methods are based, in part, upon the recognition that components for compounds can be encoded by reacting different pairs of components, each pair with a known and different molecular weight difference, to form compounds in a combinatorial library. Thus, the present approach is designed to encode the identity of pairs of library compounds; this contrasts with other methods which seek to specify the identity of single library members. Consequently, the methods provide a new approach for synthesizing combinatorial libraries in which components are self-encoded on the basis of molecular weight differences; this enables components of the compounds to be decoded, at least in part, through the use of techniques for determining molecular weight differences (e.g. mass spectrometry). The inventors refer to such methods as a “Self-Encoded Split & Pool Synthesis of Compound Multiplets.”

Hence, with certain methods of the invention, the use of paired components can be combined with other encoding strategies to provide multistep encoded synthesis schemes without concurrently using tags at one or more steps to encode the identity of the components of the library members. Alternatively, or in addition, the invention provides methods that can be combined with conventional tagging techniques to identify the identity of the components of the library members. In other methods, additional information regarding the composition of the library compounds is encoded by performing a second self-encoding step in which a second pair of components having a molecular weight difference that is characteristic for a particular pair of components is performed, and/or by tracking which component is added to each of the different reaction vessels.

II. Library Synthesis

A. Methods Generally

In general, the methods involve performing multiple synthesis cycles to synthesize compounds on a support in which components are added in a component-by-component fashion. A synthesis cycle typically involves apportioning supports into a plurality of reaction vessels or sites equivalent in number to the number of components or component pairs to be added in the cycle. The supports in the different reaction vessels are then reacted with different components of the compounds. During the reaction, the added components attach to the supports or to a component that was attached in a previous cycle. In some instances, the support includes a linker, and the components attach to the linker rather than directly to the support itself. In between most cycles, supports are pooled and then apportioned into reaction vessels for the next round of synthesis, the number of vessels being equivalent to the number of components or component pairs to be added in the next cycle.

In one of the synthesis cycles, referred to as a mixed coupling step or cycle, different pairs of components rather than single components are added to the different reaction vessels. In this way, supports within a reaction vessel receive the same pair of components and different reaction vessels receive different pairs of components. The different component pairs each have a known and distinctive difference in molecular weight that encodes for a particular component pair. Reaction of these encoded pairs with the supports or components previously attached to the support produce a population of supports that bear different pairs of compounds, each pair of compounds having a difference in molecular weight that is characteristic for the compound pair and thus encodes for the component pair added during the mixed coupling step. In this way, the identity of the members in a pair of components is “self-encoded.”

Additional cycles before and/or after the mixed coupling step can also be performed. These additional cycles can utilize various encoding schemes to encode for other components added in the synthesis of the final compounds. For example, initial components can be labeled to “pre-encode” the identity of the first (or first and second) component(s) of the compounds. Components added in the final synthesis cycle can be “spatially encoded” by generating a correspondence regime in which the identity of the final component added to each reaction vessel is tracked so that the identity of the final component of the compound is known for each of the reaction vessels. The compounds from each reaction vessels are then separately assayed for a desired activity. Another encoding option is to perform a second mixed coupling step in which second component pairs having a known difference in molecular weight are added to the supports. Methods utilizing this approach generate supports bearing at least four different compounds. The molecular weight difference between one pair of compounds encodes for the pair of components added in the first cycle using component pairs; likewise, the molecular weight difference between a second pair of compounds encodes for the pair of components added in the second mixed coupling step.

Compounds synthesized according to the methods of the invention can be screened for those compounds having a property of interest (e.g., a biological activity of interest). Compounds having the desired property or activity are isolated. The identity of the components added during synthesis cycle in which paired components were added can be determined from the molecular weight difference in two of the compounds borne by the isolated support. Other components in the isolated compounds can be determined from the other encoding schemes (e.g., see the discussion on pre-encoding and spatial encoding infra).

B. Encoding Via Mixed Coupling Step

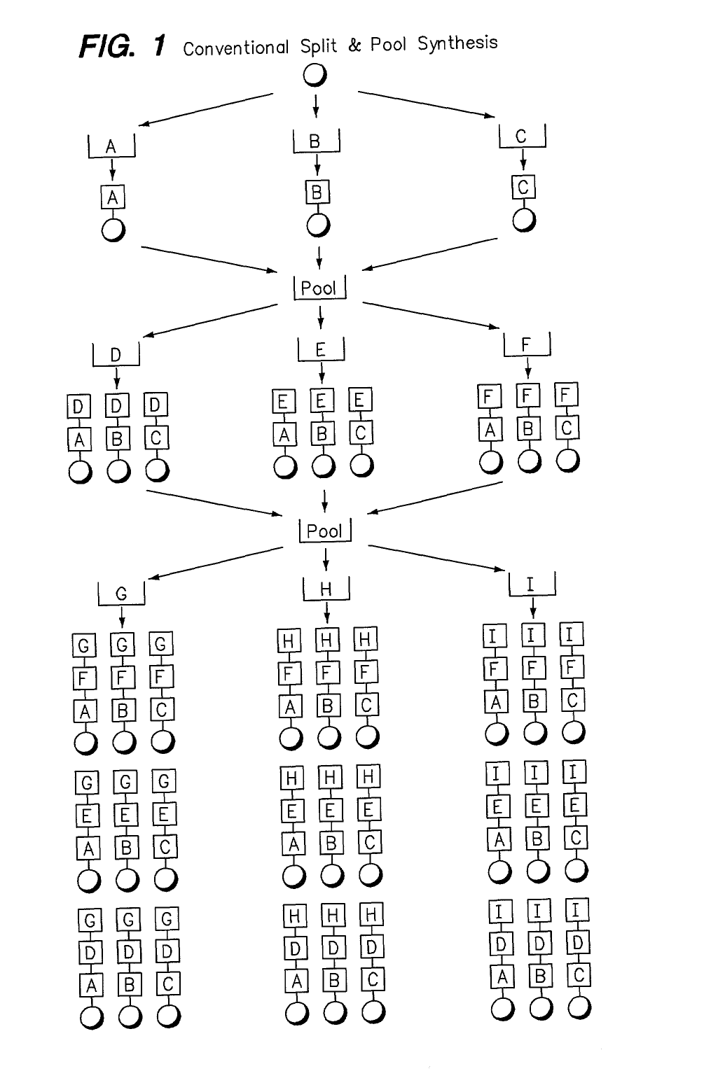

A conventional split and pool combinatorial synthesis using 3 different building blocks at each of 3 chemical steps is reviewed in FIG. 1. Initially, a population of supports are apportioned into three separate reaction vessels. Different first components (A, B and C) are attached to the supports in the three different reaction vessels. Following attachment, the supports from the three reaction vessels are pooled and then reapportioned into another three reaction vessels, where the supports in different reaction vessels are reacted in a second cycle with three different second components (D, E and F). The second components attach to the components added in the first cycle, thus forming three different nascent products in each reaction vessel. After pooling the supports, in a third synthesis cycle, the supports are again apportioned into three reaction vessels and the supports in the different reaction vessels reacted with three different components (G, H, and I). At the end of this synthesis, 3 pools of synthesis particles are formed, each pool containing 9 different products.

Because these pools are kept spatially segregated, the identity of the final building block added (i.e., “G”, “H”, or “I” in FIG. 1) is known with certainty. If one were to select any support at random from one of these pools, cleave the product from the support and obtained a mass spectrum (MS) of the selected material, one would expect to observe a molecular ion characteristic of the single compound synthesized on that particular support. Thus, if each support in the selected pool had a unique molecular weight (Mw), the identity of the compound on the selected support could be unambiguously determined. However, as noted in the Background section, molecular weight redundancy in libraries of a practical size (e.g., >100's of compounds) generally precludes an unambiguous determination from being made.

The present invention is based, in part, upon the concept that additional information about the split and pool synthesis process can be encoded if one deliberately chooses to prepare a mixture (or multiplet) of compounds on each synthesis support. Typically, this multiplet consists of 2 compounds that are produced by coupling 2 chemical building blocks at a particular step of a multiple-step split and pool process (i.e., the mixed coupling step or cycle). However, as described in greater detail below, certain methods involve coupling a second pair of chemical building blocks on a support, thereby forming 4 compounds on a support.

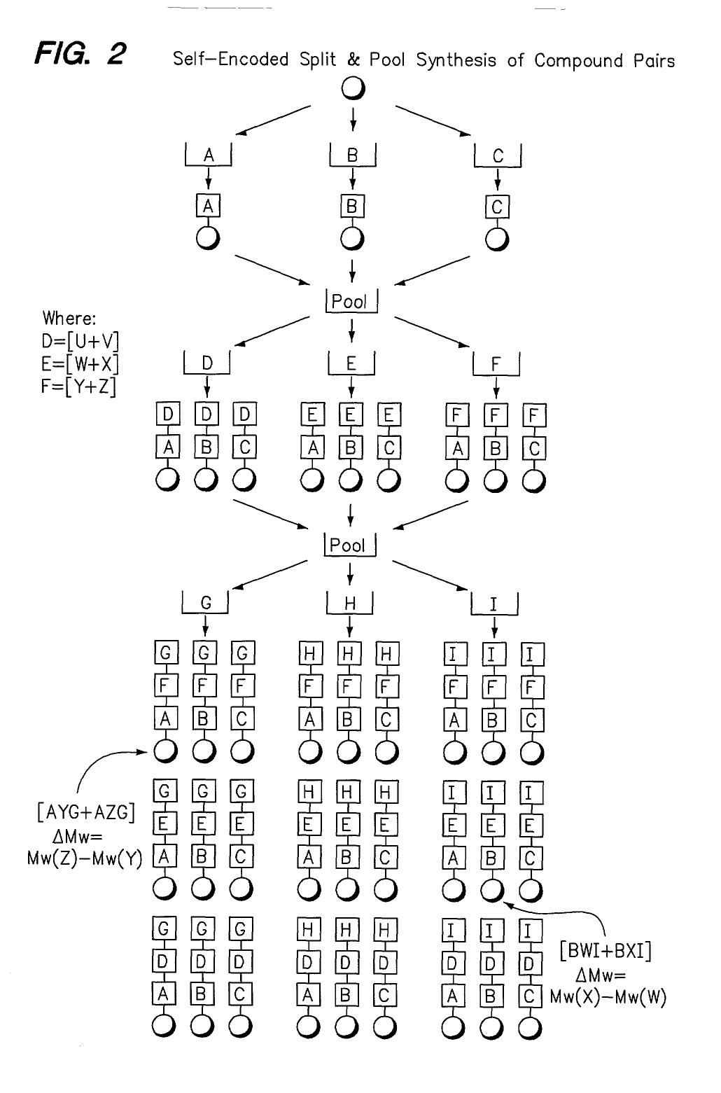

One example of such a coupling step is illustrated in FIG. 2, where two building blocks (“U” and “V”; “W” and “X”; “Y” and “Z”) are coupled to pools of supports in the second step of the synthesis. Following the reaction in the mixed coupling cycle, every support in the library bears two different products; thus, the mass spectrum on material cleaved from any single support shows 2 distinct molecular ions. For example, the supports highlighted in FIG. 2 carry the compounds [AYG, AZG] and [BWI, BXI] respectively, and thus produce 2 signals in each mass spectrum separated by a mass differential ≢Mw(Y,Z)=Mw(Z)−Mw(Y) and ΔMw(W,X)=Mw(X)−Mw(W), respectively (assuming Mw(Z)>Mw(Y) and Mw(X)>Mw(W)). By arranging each pairing of components at the mixed coupling step to give a unique mass differential, additional information about the synthesis is encoded into the mass spectrum of the compound pair.

Encoding through the use of a mixed coupling step can be formalized into the following two rules for the synthesis shown in FIG. 2:

(a) Mw(A)≠Mw(B)≠Mw(C)

(b) ΔMw(U,V)≠ΔMw(W,X)≠ΔMw(Y,Z)

When these conditions are met, the pair of mass values observed in the MS of the material cleaved from any support unambiguously specifies the identity of the 2 compounds formed on a support. The composition of the compounds or products can be determined because the absolute value of ΔMw specifies the identities of the mixed building blocks; the identity of a second building block is known through spatial encoding or pre-encoding (see below). The remaining component can be deduced by subtracting the combined molecular weight of the known components from the total molecular weight of a compound. If the material obtained from a support has an activity of interest in some type of assay (biological or otherwise), the two compounds can be resynthesized and the 2 compounds individually tested to confirm which compound is responsible for the observed activity.

More generally stated, condition (a) above means that the components whose identity is determined by subtracting the masses of all known components (e.g., as determined by the mixed coupling, spatial and/or pre-encoding methods described below) from the total molecular weight of the compounds should each have a unique molecular weight.

B. Pre-encoding

Pre-encoding generally refers to any technique by which the identity of one or more initial components in the synthesis are encoded. One form of pre-encoding involves labeling the component that is added in the first cycle, or the components added in the first and second cycles. The term label is meant to include any compound which itself is capable of being directly detected or which can generate a detectable signal. Labels include, for example, compounds that have detectable optical, electronic, magnetic or chemical properties. Thus, suitable labels include, but are not limited to, fluorophores, chromophores, radioisotopes, magnetic particles, infra-red (IR) chromophores, nuclear magnetic resonance (NMR) active nuclei and electron dense particles. The term label also includes distinctive physical characteristics of the support itself. Thus, a label can also mean, for example, the shape or size of the support, or some physical marking of the support.

Among the many pre-coding strategies available, those that use simple optical readouts (e.g., fluorescent or absorptive signatures) are particularly convenient because the encoded support can be readily imaged and decoded using an appropriate microscope or CCD-based imaging system. In one specific example, 1 μm sized fluorescent silica beads of different colors are non-covalently associated with larger polystyrene synthesis resin beads according to some predetermined binary coding scheme (see, e.g., Trau, et al. WO 99/24458, which is incorporated by reference in its entirety). This form of pre-encoding is useful since the presence and integrity of the fluorescent reporter beads is compatible with a wide range of solvents, reagents and synthetic conditions. In Example 3 below, a library of >4000 N-acyl-N-alkyl amino acid amides is prepared by using fluorescent reporter microbead pre-encoding with mixed monomer self-encoding at the third synthetic step (i.e., reductive alkylation with a mixture of aromatic aldehydes). Such microbeads are commercially available from Microbead Particle Technologies GmbH, for example.

Other specific examples include microscopically recognizable alphanumeric labels that can be attached to the support. An alphanumeric code can be used to encode a reaction step (e.g., “A1” means that component A was reacted with the support in the first reaction step). Another pre-encoding strategy utilizes molecular structures that by their composition or size (e.g., length) encode for the identity of an added component. Polynucleotides are one convenient molecular structure, as they can be readily manipulated, sequenced and amplified using a variety of known molecular biology techniques (see, e.g., Dower, et al., WO 93/06121; Lerner et al., WO 93/20242; Needels, et al. Proc. Natl. Acad. Sci. USA 90:10700-10704 (1993); and Brenner and Lemer, Proc. Natl. Acad. Sci. USA 89:5181-5183 (1992), each of which is incorporated by reference in its entirety). Peptides can also be used (see, e.g., Kerr, et al., J. Amer. Chem. Soc., 115:2529-2531 (1993); and Nikolaiev et al., Pept. Res., 6:161-170 (1993), each of which is incorporated herein by reference in its entirety). Electrophoric tags are another suitable type of label (see, e.g., WO 95/35503; Ohlmeyer et al., Proc. Natl. Acad. Sci. USA 90:10922-10926 (1993); and Still et al., WO 94/08051, each of which is incorporated by reference in its entirety).

Pre-encoding can be accomplished in various ways. For example, in some instances unlabeled supports are apportioned into multiple reaction vessels and then different first components are attached directly to the support (or optionally via a linker). Before pooling the supports, the supports are reacted with a label to form labeled supports. In this approach, different labels are added to each reaction vessel, thereby making it possible to determine the identity of the first component of a compound by identifying the label associated with the support. For example, three compounds, A, B and C, are reacted with unlabeled supports in separate reaction vessels 1, 2, and 3, respectively. Subsequently, a first label, a second label and a third label are placed into reaction vessels 1, 2, and 3, respectively, where they attach to the supports within the particular reaction vessel. If an isolated compound found through an assay to have a desired property bears the second label, this indicates that the first component of the compound is component B (i.e., the first component added in the second reaction vessel). Of course, the order of labeling can be reversed such that the supports in the different reaction vessels are distinctively labeled before a component is attached. In yet another approach, pre-labeled supports are apportioned into the different reaction vessels, each reaction vessel receiving a plurality of supports bearing the same label, but different reaction vessels receiving different labeled supports. By using pre-labeled supports, a separate labeling step is not necessary.

C. Spatial Encoding

Spatial encoding refers to processes in which a correspondence regime is created such that the identity of the final component added to the different reaction vessels is tracked. Thus, with spatial encoding the identity of the final component in each reaction vessels is known. In methods utilizing spatial encoding, the pools of different compound-bearing supports in the different reaction vessels are kept spatially segregated and are not pooled after the final reaction step. Thus, assays are not performed with aliquots containing multiple different compound-bearing supports, but with separate aliquots from individual reaction vessels. By keeping the reaction vessels segregated in this way and by separately withdrawing aliquots for separate assays, it is possible to track, and thus identify, the final component of compounds which give positive assay results.

D. Three Step Syntheses

One example of a three step synthesis has been described above in the discussion concerning the mixed coupling step. However, a variety of different 3-cycle synthesis schemes can be developed by choosing different combinations and orders of encoding strategies. For example, in certain formats, the mixed coupling cycle is the second cycle in which the second component is reacted with the supports. The identity of the first component can be encoded by using labels or other types of pre-encoding. In such instances, the identity of the component added in the third cycle can be determined from the total molecular weight of a compound less the total weight of the first component (known from labeling) and the second component (known from the molecular weight difference between the paired compounds formed on a support).

In other formats, the identity of the third component rather than the second component is encoded. The third component is spatially encoded as described above by tracking which final component is added to each reaction vessel; in this way, the identity of the final component is known for each reaction vessel. For 3-step methods in which the final component is spatially encoded, the identity of the first component can be identified by subtracting the combined weight of the component added in the second step (known from the molecular weight difference between the paired compounds on the support) and the third step (known from spatial encoding) from the total weight of the compound.

All steps can be encoded by pre-encoding the first component, using a mixed coupling step to encode the second component and by spatially encoding the third component. Since all the steps are encoded, it is not necessary to subtract the combined weight of two components from the total weight of a compound to determine the identity of one of the components.

In still other methods, the mixed coupling cycle is performed first and provides the first component of the final compounds. In such methods, the final component is typically spatially encoded. The unknown component can be determined by subtracting the combined molecular weight of the first component (known from the molecular weight difference between the paired compounds on the support) and the third component (known from spatial encoding) from the total molecular weight of the compounds.

The mixed coupling step can also be performed during the third synthesis cycle in which the final component is added. The first component added during the first cycle is then encoded by a pre-encoding technique. The remaining component added during the second cycle can be determined from the molecular weight difference between the total molecular weight of the compounds and the combined weight of the first and third components (known from pre-encoding and the molecular weight difference of the paired compounds, respectively).

E. Four Step Syntheses

The self-encoding strategy utilizing a mixed coupling step can be extended to higher order combinatorial syntheses. For example, the invention provides a variety of 4-step combinatorial synthesis methods. Most typically, such methods involve a self-encoding step (i.e., mixed coupling step) in combination with pre-encoding and spatial encoding. In a 4-step synthesis, the first coupling step is usually pre-encoded (e.g., supports are labeled). By reserving spatial encoding for the fourth synthetic step and using mixed coupling at either of the second or third steps, the mass spectrum of the product from any bead can be used with the pre-encoding information to unambiguously specify the reaction history of the 2 products on any given support. Thus, for example, if the first step is pre-encoded, the second step is encoded by mixed coupling and the fourth component is spatially encoded, then the identify of the third component can be determined from the total molecular weight of an active compound less the combined molecular weight of the encoded components.

In certain other methods of the invention, two steps in a 4-step combinatorial synthesis involve the addition of monomer pairs, producing supports that contain 4 distinct products and thus giving rise to 4 molecular ions in the MS. In these methods, the pattern of 4 ions observed is indicative of the building blocks incorporated. For example, addition of components A (first step); B and B′ (first component pair added in first mixed coupling step), C and C′ (second component pair added in second mixed coupling step) and D (fourth step), result in the following four compounds being formed on a support: 1) A-B-C-D, 2) A-B′-CD, 3) A-B-C′-D and 4) A-B′-C′-D. The identity of the first component pair (B and B′) can be determined from the molecular weight difference between one pair of compounds (e.g., compounds 1 and 2); similarly, the identity of the second component pair (C and C′) can be determined from the molecular weight difference between a second pair of compounds (e.g., compounds 1 and 3, or compounds 2 and 4). However, the resulting decrease in quantity of each product available for testing, plus the requirement to resynthesize and test 4 separate compounds to fully identify the active compound complicates this approach somewhat.

If two mixed coupling steps are utilized, at least the first or fourth step is typically also encoded so that that the identity of all the components can be identified. If the first step is pre-encoded by labeling for example, the fourth component can be deciphered from the total molecular weight of the compound minus the combined weight of the first component (encoded by label), second component (encoded by mixed coupling) and the third component (encoded by mixed coupling). In like manner, when the fourth component is spatially encoded, the first component can be determined from the total molecular weight of a compound less the combined weight of the second component (encoded by mixed coupling), third component (encoded by mixed coupling) and fourth component (encoded spatially). Of course, all steps can be encoded if the first step is pre-encoded, the second and third steps are encoded by mixed coupling and the final step is spatially encoded.

F. Five-step Syntheses

By pre-encoding the first two diversity steps of a synthetic protocol, the preencoding/self-encoding method can be utilized to track a 5 diversity step synthesis. Such methods typically involve separately and distinctively pre-encoding n aliquots of synthesis particles, where n is the product of the number of building blocks to be used at the first and second steps (i.e., n=A×B, where A=no. of first building blocks and B=no. of second building blocks). Parallel synthesis is then used to prepare these n different “dimer” products, before pooling and performing the remaining three synthetic steps according to the split and pool paradigm described above for the three step synthesis in which one cycle is self-encoded by using mixed coupling (either the third or fourth step of a five step synthesis) and the components reacted in the final step are spatially encoded.

In certain methods, parallel synthesis generally includes initially apportioning supports into multiple reaction vessels (A in number) and reacting the supports in the different reaction vessels with different first components. Aliquots from each of the reaction vessels are then removed and divided into a plurality of equal portions. The number of portions (B) is equivalent to the number of different components to be utilized in the second step. As a consequence of the splitting of a pool, supports from any given reaction vessel are placed into multiple reaction vessels (B in number), thereby forming a total of n (A×B) reaction vessels.

To illustrate, if five components/building blocks are used in the first step (A=5), supports are initially apportioned into five reaction vessels. The supports in these five reaction vessels are then reacted with the five different components, each reaction vessels receiving a different component. If two different components (B=2) are added in the second step, then an aliquot is withdrawn from each of the five reaction vessels, divided into two equal portions (a first and second portion) and the two portions placed into separate reaction vessels. As a result, a total of 10 reaction vessels (n=A×B=10) contain supports. The first portion taken from each reaction vessels is reacted with one of the two components to be added in the second cycle; the second portion is reacted with the other component. After adding the components in the second cycle, the supports from the all the reaction vessels (n in number) are pooled and then apportioned into a plurality of reaction sites, the number of reaction sites equivalent to the number of components to be utilized in the third synthesis cycle. As noted above, the remaining three cycles are performed according to the procedure described above for a 3-step synthesis in which the identity of a component is self-encoded via a mixed coupling step (step 3 or 4 of a five step method) and the final step is spatially encoded (step 5 of a five step method).

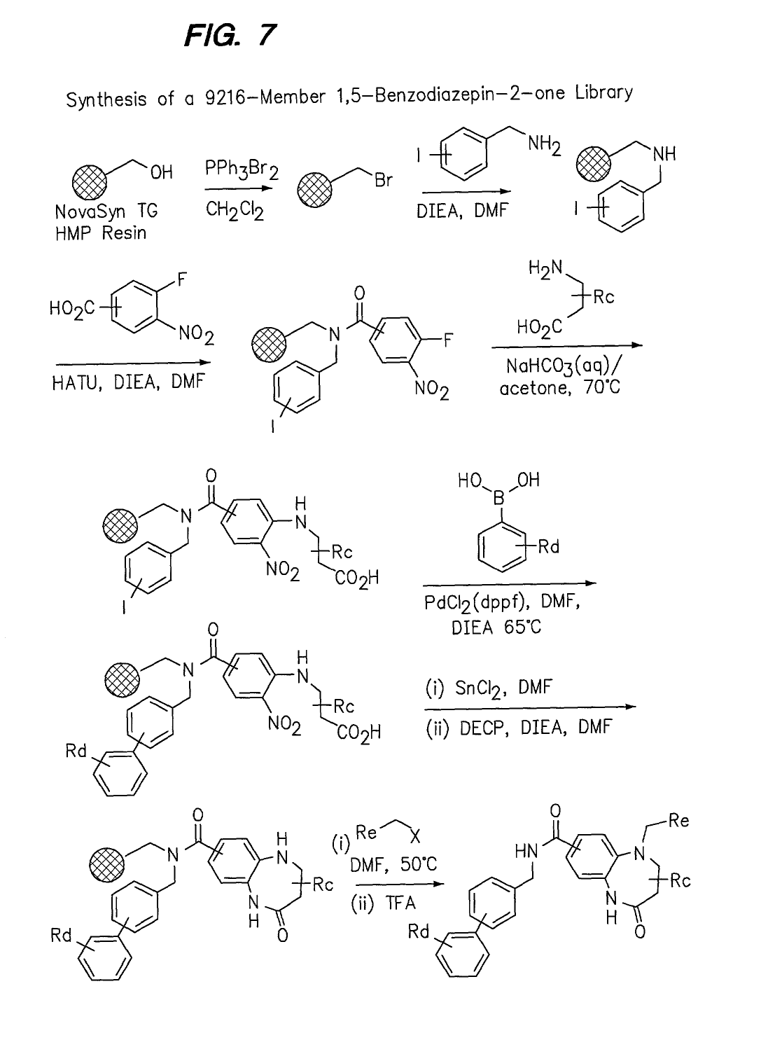

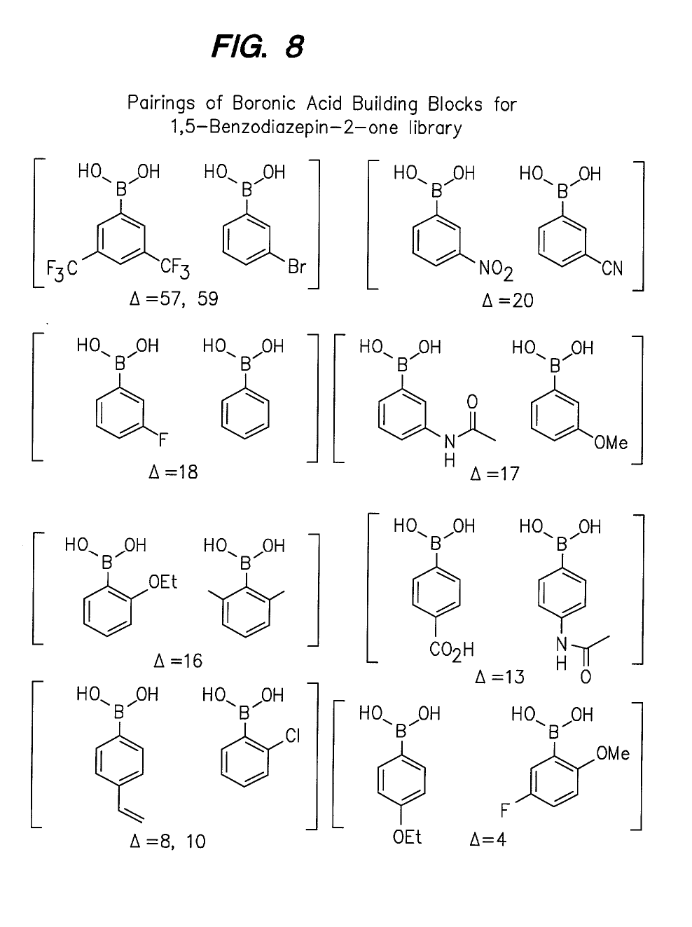

This type of approach is exemplified by the 5-step synthesis of 1,5-benzodiazepin-2-ones in Example 4 below (see FIG. 7; for a discussion of the synthesis of 1,5-benzodiazepin-2-ones, see Schwarz et al., (1998) Tetrahedron Lett. 39: 8397). This reaction sequence features Suzuki-type cross coupling of polymer-supported aryl iodides with boronic acids (see, e.g., Ruhland et al., (1997) J. Org. Chem. 62: 7820) where the boronic acid building blocks are paired as shown in FIG. 8. Because of the natural isotopic pattern of halogen atoms (Br and Cl) found in some of the building blocks employed in this library, further “multiplet structure” is expected in the mass spectrum beyond the pair of molecular ions found for products in the previous examples.

In certain other methods, parallel synthesis involves using as many aliquots of supports for each of the first coupling steps as there are components to be added at the second coupling step, i.e., the total number of reaction vessels into which the supports are initially apportioned is equal to the number of components to be added in the first step (A) multiplied by the number of components to be added in the second step (B). For example, if five components/building blocks are to be added at the first coupling step (A=5) and four components/building blocks are to be added in the second synthesis step (B=4) then initially supports are apportioned into a total of twenty reaction vessels. In the first synthesis cycle, the five different first components are added to the apportioned supports in the twenty reaction vessels, the number of reaction vessels to which any particular first component is added being equal to the number of components to be added in the second synthesis cycle. Hence, in this example, each of the five different first components is added to four reaction vessels. In the second synthesis cycle, supports in different reaction vessels that were reacted with the same first component are reacted with different second components. After the supports have been reacted with the second components, the supports from all the reaction vessels are pooled and then apportioned into a plurality of reaction sites, the number of reaction sites being equivalent to the number of components to be added in the third synthesis cycle. The remaining steps (third through fifth synthesis cycles) are as described above for a 3-step synthesis using self-encoding at step 3 or 4 and spatial encoding for the final step (see supra).

G. Mixed Coupling Step

The mixed coupling step can be performed in various ways. The most straightforward approach is to treat the reactants borne on a support with a physical mixture of the building blocks under standard conditions that promote the given reaction. It should be appreciated, however, that different monomers can in some instances undergo coupling reactions at different rates, and that in instances where it is important to achieve approximately equimolar representation of the two products on each support, the concentrations of the reactants may need to be adjusted appropriately (e.g., biasing the ratio of monomer concentrations in favor of the less reactive building block).

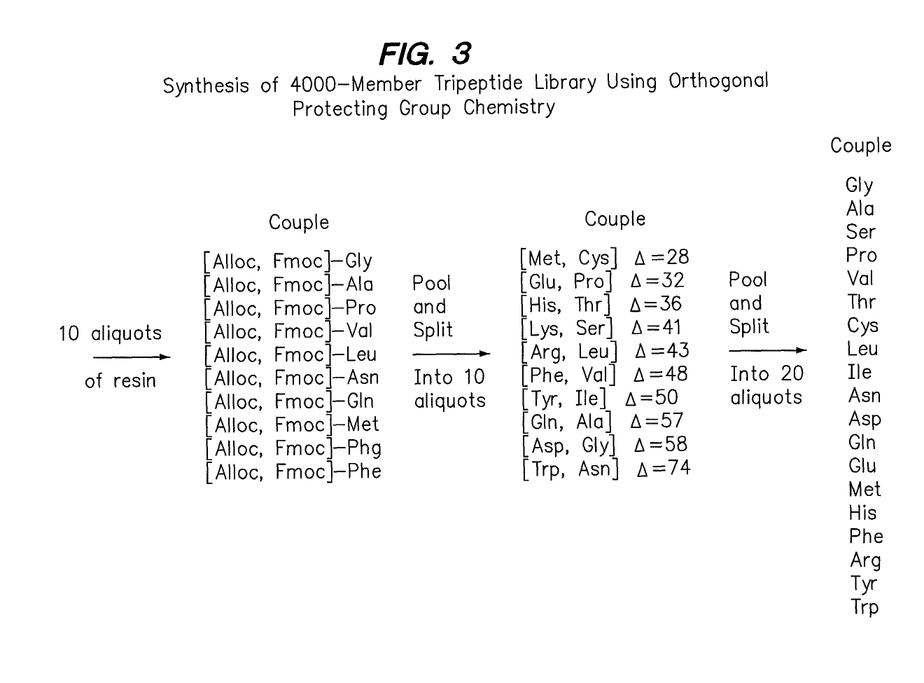

A second approach is to employ orthogonal protecting group chemistry with one set of particle-supported reactants. This can be conveniently achieved when the building blocks are α-amino acids, as both Fmoc and Alloc-protected monomers are widely available or readily prepared. This is illustrated in Example 1 below (see also FIG. 3), wherein a 4000 -member tripeptide library is prepared by: (i) first coupling an equimolar mixture of 10 different Alloc and Fmoc protected amino acids to photolabile resin; (ii) pooling and splitting the resin into 10 aliquots; (iii) treating each aliquot with piperidine to remove the Fmoc groups and then coupling the first of a preselected pair of Fmoc-protected amino acids to each aliquot; (iv) treating the aliquots with [Bu₄N][N₃] in the presence of catalytic Pd to remove the Alloc groups and then coupling the second of the pair of Fmoc-protected amino acids to each aliquot; (v) pooling and splitting the resin into 20 aliquots; (vi) treating each aliquot with piperidine to remove the Fmoc groups; (vii) coupling one of 20 different Fmoc-protected amino acids to each aliquot; (viii) deprotecting each resin aliquot with TFA. A potential shortcoming of this method, however, is that in some instances it can be difficult to arrange protecting group chemistry such that one half of the product on each particle can be elaborated independently of the other half. Accordingly, the mixed coupling protocol mentioned earlier is more practical in these instances.

In general terms, the building blocks/components can be paired according to a variety of different parameters or criteria, provided a unique mass differential (ΔMw) is maintained for each pair. In some instances, however, it is useful to favor specific pairings. For example, building blocks with similar steric and/or electronic properties can react with the particle-supported reagents at similar rates and can be combined to form “isokinetic” building block pairs. Thus, the term “sterically similar” means that the components have related steric structures such that the components react at similar rates to produce compounds in substantially the same concentration. Likewise, the term “electronically similar” refers to components having sufficiently related electronic characteristics (e.g., charge and/or polarity) that the components react to form compounds at substantially the same rates and thus yield compounds that have substantially the same concentration on the support. Typically, the concentrations of compounds borne by a support are considered substantially the same if the relative concentrations are within 200 percent; in other instances, within 100 percent, in still other instances within 50 percent, and in yet other instances the relative concentrations are within 20 percent.

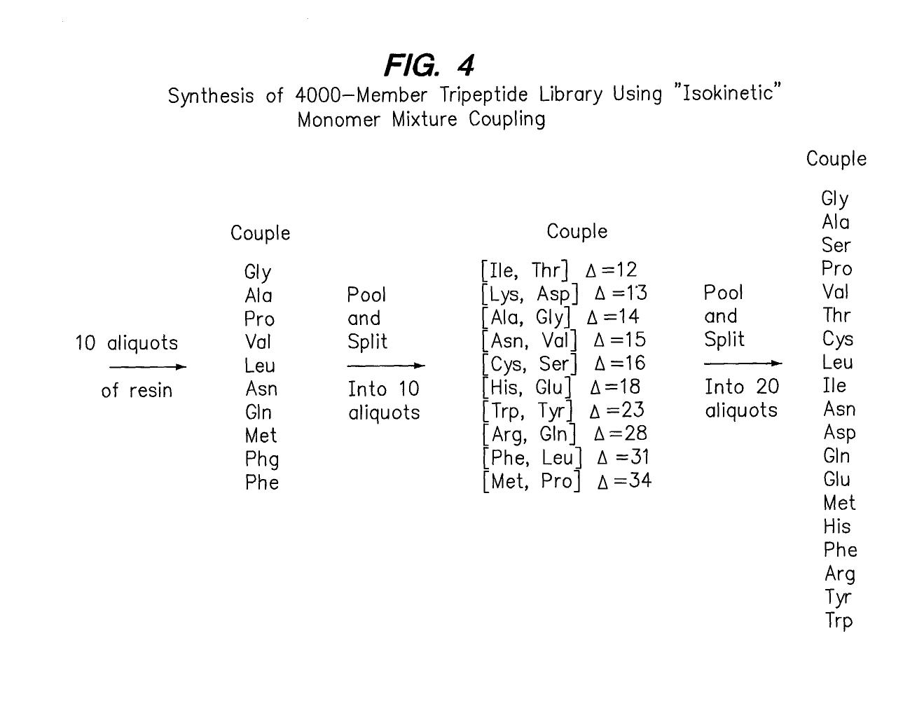

The relative coupling rates of the common α-amino acids have been determined (see, e.g., Eichler, et al.,(1993) Biochemistry 32: 11035). This data has been used in Example 2 below to devise an alternative “isokinetic” pairing scheme (see FIG. 4) for the synthesis of the same 4000-member tripeptide library as described in Example 1 and illustrated in FIG. 3. In another useful pairing strategy, isokinetic monomer mixtures are formed which have either similar or dissimilar physicochemical properties (i.e., chemical properties of a compound that effect its physiological properties, e.g., charge or polarity). Utilizing such a strategy, it is possible to ensure that if some activity (e.g., biological) is observed for the compounds borne by a given support that the activity is more (or less) likely to result from the cumulative activity of both compounds borne by the support.

H. Reactive Coupling

The methods of the invention initially begin with the apportioning of a plurality of supports. Typically, the supports are divided into as many reaction vessels as there are different components to be added in a reaction step. The number of supports used generally depends upon the total number of different compounds to be synthesized multiplied by the number of library equivalents (i.e., the average number of supports carrying each type of compound) to be prepared. A variety of different types of reaction vessels can be utilized including, but not limited to, microtiter wells, columns, flasks and other standard containers utilized for organic synthesis. After a synthesis cycle, the supports are typically pooled and then reapportioned into another group of reaction vessels, the number of reaction vessels into which the supports are apportioned again being equivalent to the number of different building blocks being utilized in the particular synthesis cycle.

Attachment of the different components can be achieved utilizing chemical, enzymatic, or other means, or combinations thereof. In general, the methods of the invention can employ essentially any synthetic method including, but not limited to, synthetic methods for preparing diverse heterocyclic, and/or carbocyclic and/or oligomeric molecules. Synthetic strategies for joining components varies according to the nature of the components being joined. Synthetic strategies for coupling components from the same or different families (e.g., nucleotides, amino acids and carbohydrates) are well-established. For example, phosphoramidite or phosphite chemistries can be employed when coupling nucleotides. For polypeptides, coupling and blocking strategies (e.g., Fmoc, Alloc or Boc chemistries) are well-known (see, e.g., The Peptides. vol. 1 (Gross, E. and Meienhofer, J., Eds.), Academic Press, Orlando (1979)), which is incorporated by reference in its entirety for all purposes).

Different components can attach directly to the support or to the support via one or more components added in any of the preceding synthesis cycles. Hence, it is possible to form compounds that are linear, branched, cross-linked and/or cyclic in structure.

The number of different components being reacted in any given step can be expanded or contracted. For example, one step can involve apportioning the supports into 5 different reaction vessels for reaction with 5 different components. The next step, however, can involve pooling the supports and apportioning the supports among 10 different reaction vessels for reaction with 10 different components. The components added in the different steps can be of the same type or can be different and can be coupled according to chemistries described in the foregoing references.

I. Library Composition

1. Compounds

The compounds borne by the supports can be composed of any components that can be joined to one another through chemical bonds in a series of steps involving the addition of different components at each step. Thus, the components can be any class of monomer useful in combinatorial synthesis. Hence, the components, monomers, or building blocks (the foregoing terms being used interchangeably herein) can include, but are not limited to, amino acids, carbohydrates, lipids, phospholipids, carbamates, sulfones, sulfoxides, esters, nucleosides, heterocyclic molecules, amines, carboxylic acids, aldehydes, ketones, isocyanates, isothiocyanates, thiols, alkyl halides, phenolic molecules, boronic acids, stannanes, alkyl or aryl lithium molecules, Grignard reagents, alkenes, alkynes, dienes and urea derivatives. The type of components added in the various steps need not be the same at each step, although in some instances the type of components are the same in two or more of the steps. For example, a synthesis can involve the addition of different amino acids at each cycle; whereas, other reactions can include the addition of amino acids during only one cycle and the addition of different types of components in other cycles (e.g., aldehydes or isocyanates).

Given the diversity of components that can be utilized in the methods of the invention, the compounds capable of being formed are equally diverse. Essentially molecules of any type that can be formed in multiple cycles in which the ultimate compound or product is formed in a component-by-component fashion can be synthesized according to the methods of the invention. Examples of compounds that can be synthesized include polypeptides, oligosaccharides, polynucleotide, phospholipids, lipids, benzodiazepines, thiazolidinones and imidizolidinones. As noted above, the final compounds can be linear, branched, cyclic or assume other conformations. The compounds can be designed to have potential biological activity or non-biological activity.

The number of compounds formed depends upon the number of different components utilized in the various steps. The number of members in the library can be as few as two; however, typically there are many more members, including 10², 10⁴, 10⁶, 10⁸, 10¹⁰, 10¹² or 10¹⁵ members, or any number of members therebetween. As used here, the term member refers to each distinct compound borne by a support, not the pair of compounds borne by the support.

2. Supports

The materials upon which the syntheses of the invention are performed are interchangeably referred to herein as supports, particles or beads, for example. These terms are generally meant to include materials that are capable of supporting the growth of a compound formed through repetition of multiple synthetic cycles and compatible with the different types of chemical reactions performed in the synthesis of such compounds.

The terms include, but are not limited to, solid supports such as organic polymeric supports (e.g., cellulose beads, polystyrene beads, polyacrylamide beads and latex beads) and supports composed of inorganic materials (e.g., pore-glass beads, silica gels and metal particles). Often the organic polymeric support materials are cross-linked to provide additional stability. The supports can be of a variety of different shapes, including for example, disks, capillaries, spheres, ellipsoids and the like.

The size of the support is chosen such that the support is sufficiently large so that the paired compounds and optional label and/or reporter can readily be attached thereto. In general, the solid support size is in the range of 1 nm to 500 microns in diameter; more typically, the supports range from less than 10 microns to about 500 microns in diameter. In certain applications the supports are only about 10 nm to about 200 nm in diameter. A more TM massive support of up to 1 mm in size can sometimes be used. MONOBEADS™ (Pharmacia Fine Chemicals AB, Uppsala Sweden) TentaGel (Rapp Polymere), ArgoGel (Argopnaut Technologies) or their equivalent are examples of commercially available supports that can be used.

Depending upon the type of support, the support can naturally contain a variety of surface groups to facilitate attachment of the first components of the compounds, such as hydrophilic, ionic or hydrophobic groups. For example, the support can include one or more chemical functional groups to enhance attachment (e.g., hydroxyl, amino, carboxyl and sulfhydryl). Alternatively, the support can be derivatized to add such functional groups. These functional groups are also useful for the attachment of the optional linkers to which the components can attach and/or the optional labels used for pre-encoding an initial step in the synthesis.

Nanoparticles are one type of support that is useful with certain methods of the invention. Nanoparticles suitable for use in the invention can be prepared from a variety of materials, such as cross-linked polystyrene, polyesters and polyacrylamides or similar polymers. For use in vivo, biodegradable nanoparticles are particularly preferred. Such particles may be prepared from biocompatible monomers as homopolymers or as block copolymer materials. Examples of such polymers include, but are not limited to, polylactic acid, polyglycolic acid, polyhydroxybutyric acid and polycaprolactone, polyanhydrides and polyphosphazenes. When used in cellular transport assays (see infra), frequently the particles are fabricated to contain an exterior surface comprising a hydrophilic polymer such as poly(alkylene glycol), poly(vinyl alcohol), polysaccharide or polypyrrolidine to resist uptake of the particles in vivo by the reticuloendothelial system. Such particles are described in U.S. Pat. Nos. 5,578,325 and 5,543,158, which are incorporated by reference in their entirety for all purposes.

The nanoparticles can be synthesized according to several known methods (see, e.g., U.S. Pat. No. 5,578,325) or can be purchased from commercial suppliers such as Polysciences and Molecular Probes. The nanoparticles can be labeled with fluorescent molecules, and such nanoparticles are commercially available from Molecular Probes, for example. Nanoparticles can be prepared from block copolymers by emulsion/evaporation techniques using the pre-formed copolymer. With such techniques, polymer is dissolved in an organic solvent and emulsified with an aqueous phase by vortexing and sonication (higher energy sources giving smaller particles). The solvent is evaporated and the nanoparticles collected by centrifugation.

Other suitable supports include, for example, molecular scaffolds, liposomes, (see, e.g., Deshmuck, D. S., et al., Life Sci. 28:239-242 (1990); and Aramaki, Y., et al., Pharm. Res. 10:1228-1231 (1993)), protein cochleates (stable protein-phospholipid-calcium precipitates; see, e.g., Chen, et al., J. Contr. Rel 42:263-272 (1996)), and clathrate complexes. Dendrimers can also be used in some applications; these compounds can be synthesized to have precise shapes and sizes and to include a variety of surface groups (e.g., hydrophilic, ionic or hydrophobic) to facilitate attachment of components, labels and/or reporters (see, e.g., Tomalia, D. A., Angew. Chemie Int. Edn. 29:138-175 (1990); and Sakthivel, T., et al., Pharm. Res. (Suppl) 13:S-281 (1996)). Each of the foregoing publications is incorporated by reference in its entirety for all purposes.

3. Linkers

In some instances, the compounds are connected to the support via a linker. This enables the compounds to be released from the support prior to conducting assays for an activity of interest. The linkers typically are bifunctional (i.e., the linker contains a functional group at each end that is reactive with groups located on the support and the component to which the linker is to be attached); the functional groups at each end can be the same or different. Examples of suitable linkers include, but are not limited to, straight or branched-chain carbon linkers, heterocyclic linkers and peptide linkers. Exemplary linkers that can be employed in the present invention are available from Pierce Chemical Company in Rockford, Ill. and are described in EPA 188,256; U.S. Pat. Nos. 4,671,958; 4,659,839; 4,414,148; 4,669,784; 4,680,338, 4,569,789 and 4,589,071, and by Eggenweiler, H. M, (1998) Drug Discovery Today, 3: 552.

The choice of linker depends on whether the linker is intended to remain permanently in place or is intended to be cleaved so as to release the compounds borne by the support before the compounds are assayed. If a cleavable linker is desired, NVOC (6-nitroveratryloxycarbonyl) linkers and other NVOC-related linkers are examples of suitable photochemical linkers (see, e.g., WO 90/15070 and WO 92/10092), as are nucleic acids with one or more restriction sites, or peptides with protease cleavage sites (see, e.g., U.S. Pat. No. 5,382,513). Suitable supports having photochemical linkers include Hydroxymethyl Photolinker AM resin from Novabiochem, for example. Such a linker should be stable under the relevant synthesis conditions, but should allow release of the test compound in the course of the assay.

4. Reporter

In some of the assays utilized in the methods of the invention, it is helpful for the support to include a reporter to detect supports which bear active compounds. In general terms the reporter is any compound capable of being directly detected or capable of forming a detectable signal during an assay to identify compounds having a desired property. Examples of suitable reporters include, for example, chromophores, fluorophores, radioisotopes, magnetic particles, electron dense particles and a substrate for an enzyme. The reporter can be added at any step during the synthesis of the compound or can be added after the completion of the synthesis cycles. The reporter contains appropriate functional groups (or can be derivatized to contain such functional groups) to facilitate attachment of the reporter to a support. In some instances, the label attached to the support to encode for a component of added during the synthesis can serve as the reporter.

IV. Screening for Desired Property

Once formed, the combinatorial libraries of the invention can be used to screen for a property of interest. The property of interest can be any chemical, electrical, structural or biological property of interest. In many instances, the libraries are screened to identify new compounds that have some type of biological activity of interest. Specific examples of biological activities include, but are not limited to, ability to bind to a receptor, ability to agonize or antagonize a receptor, ability to bind to a receptor and trigger signal transduction, ability of protein to bind to a particular nucleic acid sequence, capacity to be transported through a cell, capacity to be an inhibitor or substrate for an enzyme and capacity to kill microorganisms (e.g., bacteria, viruses, fungi). However, compounds can be screened for other types of activity (i.e., non-biological activity) as well. For example, compounds can be synthesized to potentially have catalytic activity, or to have a desired conductivity, resistivity, or dielectric property.

Screening of the compounds of the library can be performed with the compound-bearing supports. More typically, however, the compounds are cleaved from the support to allow for less hindered interaction between the compound and target (e.g., receptor or cell). If the compounds are cleaved from the support prior to conducting the assay, however, a sample of the compound-bearing supports or an aliquot of material cleaved from the supports must be retained for use in determining the molecular weight of the compounds borne by the support as part of the decoding process.

A. Receptor Binding Assays

1. Direct Binding Assay Using Labeled Compound

One approach for screening library compounds for those capable of binding a particular receptor involves attaching a reporter to a compound or compound-bearing support (if the support bears a label from a pre-encoding step, that label often can serve as the reporter) to aid in detection of binding to a receptor of interest. For example, a receptor of interest (or a cell expressing the receptor of interest) can be immobilized on a solid support according to known procedures. An aliquot of a pair of labeled compounds, or supports bearing a pair of compounds, is withdrawn from a reaction vessel and contacted with the immobilized receptor under conditions conducive to specific binding. Unbound compound is rinsed away. Binding of compound to the immobilized receptor can be detected by detecting labeled compound or compound-bearing support bound to the solid support to which the receptor is attached. Such assays are typically conducted using multi-well plates, in which each well contains the immobilized receptor of interest.

The general method just described can be modified for multiplex analysis. In such assays, multiple different receptors are placed in a single assay location (e.g., a well in a multi-well plate) so that binding of compounds to multiple different receptors is assayed simultaneously. In certain multiplex methods, each of the different receptors is attached to a different type of solid support, each type of solid support being distinguishable from the other support types. For instance, the solid supports may differ in size, shape or be labeled with different labels (e.g., different fluorescent dyes). Confocal or semi-confocal microscopy can distinguish between the different support structures and thus can identify which of the receptors is bound to a compound. The confocal and semi-confocal fluorescent microscopy equipment necessary to conduct such assays is commercially available from either Perkin Elmer (FMAT instrument) or Cellomics, for example.

2. Direct Binding Assay Using Labeled Receptor (e.g., via FACS)

Another option for assaying for receptor binding is to contact the compound-bearing supports with fluorescently labeled receptors. The compounds are allowed to form a complex with the receptors and then washed to remove unbound or non-specifically bound receptors. Some type of confocal imaging system (as above) can then be utilized to identify compound-bearing supports to which a fluorescent receptor is bound. Alternatively a FACS instrument can be utilized to identify and physically isolate compound-bearing supports to which a fluorescent receptor is bound.

3. Competition Binding Assay

A third type of assay is a competition binding assay. A compound known to bind to the receptor at a functional site is labeled with a reporter. Such a labeled ligand may be referred to as the “tracer”. The test compounds, usually after cleavage from the synthesis supports, are added, along with the tracer to an immobilized form of the receptor. A parallel incubation of the tracer alone plus immobilized receptor is also performed. After an appropriate time, unbound compounds are washed away and the amount of tracer remaining bound to the receptor is quantified. The method of detection of bound tracer is dependent on the nature of the label and includes radioactive counting, fluorescence detection, optical imaging, luminescence, colorimetry, and the like. The ability of the test compound(s) to inhibit binding of the tracer to the receptor is taken as evidence of binding of the test compound(s) to the receptor.

B. Assays for Cellular Transport

1. General

The compounds of the libraries of the invention can also be assayed to identify compounds that are capable of being transported into or through a cell. Although a summary of how such assays can be conducted is provided below, further details regarding such assays are set forth in copending U.S. Application No. 60/154,071, filed Sep. 14, 1999, and copending U.S. application Ser. No. 09/309,174, filed May 10, 1999, and U.S. application Ser. No. 09/661,927, filed Sep. 14, 2000, each of which are incorporated by reference in their entirety for all purposes.

Active transport of compounds into or through cells typically occurs by carrier-mediated systems or receptor-mediated systems. Carrier-mediated systems are effected by transport proteins anchored to the cell membrane and function by transporting their substrates by an energy-dependent mechanism. In receptor-mediated transport systems, substrate binding triggers an invagination and encapsulation process that results in the formation of various transport vesicles to carry the substrate into and through the cell.

2. In vitro assays

For in vitro assays for transport activity, typically the compound-bearing support(s) also include some type of reporter capable of generating an optical signal. The reporter is typically attached to the support (either directly or via a linker). The methods generally involve contacting one or more cells expressing one or more transporter proteins with compounds from a library of the invention. After incubating for a period of time sufficient to permit transport or binding of the compounds, the location of signal from the reporter is detected. Detection of the signal within the cell or at a location that evidences that a complex has passed through a cell, indicates that the support bears a compound that is a substrate for a transport system expressed by the cell.

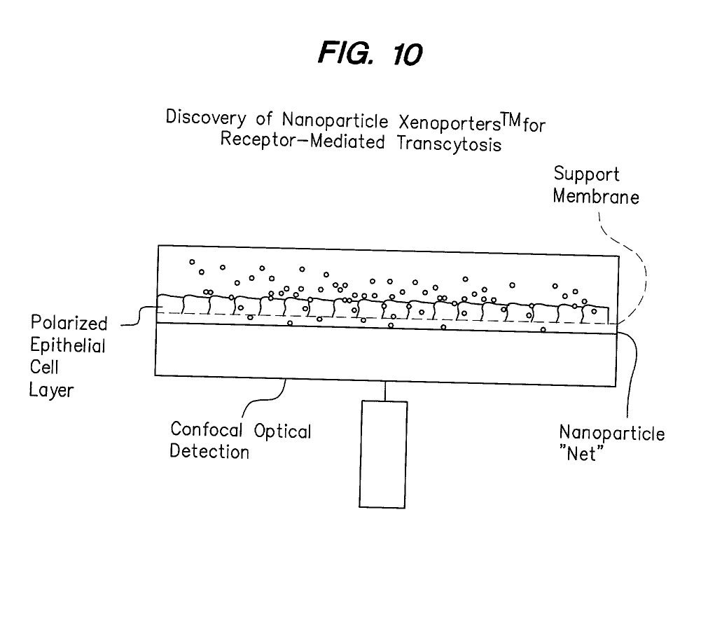

One assay method designed especially to screen for compounds capable of being transported through a cell utilizes a two membrane system (see FIG. 10). The first membrane or upper membrane is a porous membrane that includes pores that are larger than the compound-bearing support(s) being screened. A monolayer of polarized cells is placed on this upper membrane. A second or lower porous membrane is positioned under the first membrane and is structured to retain any complexes capable of traveling through the polarized cells and through the pores in the upper membrane. Porous membrane systems are available from Corning Costar and are sometimes called “transwells.”

Internalization of a compound or compound-bearing support can be detected by detecting a signal from within a cell from any of a variety of reporters. The reporter can be as simple as a label such as a fluorophore, a chromophore, a radioisotope, a magnetic particle or an electron dense reagent. The reporter can also be a protein, such as green fluorescent protein or luciferase attached to a compound or compound-bearing support. Confocal imaging can also be used to detect internalization of a compound or compoundbearing support as it provides sufficient spatial resolution to distinguish between fluorescence on a cell surface and fluorescence within a cell; alternatively, confocal imaging can be used to track the movement of compounds or compound-bearing supports over time. In yet another approach, internalization of a compound is detected using an attached reporter that is a substrate for an enzyme expressed within a cell. Once the complex is internalized, the substrate is metabolized by the enzyme and generates an optical signal that is indicative of uptake. Light emission can be monitored by commercial PMT-based instruments, by CCD-based imaging systems or by confocal microscopy.

Movement of compounds or compound-bearing supports through the layer of cells on the transwell system described above can be observed with confocal microscopy, for example. Alternatively, movement of packages through cells can be monitored using a reporter that is a substrate for an enzyme that is impregnated in a membrane supporting the cells. Passage of a support bearing such a substrate generates a detectable signal when acted upon by the enzyme in the membrane. This assay can be performed in the reverse format in which the reporter is the enzyme and substrate is impregnated in the membrane.

3. In vivo assays

The compound-bearing supports synthesized by the methods of the invention can also be used in in vivo screening methods to identify compounds that are substrates for transport proteins. In general, the in vivo methods involve introducing a compound or compound-bearing support (typically a population of such supports) into a body compartment in a test animal and then recovering those compounds or compound-bearing supports that are transported through cells lining the body compartment into which the supports were placed. More specifically, the screens typically involve monitoring a tissue or body fluid (e.g., the mesenteric blood and lymph circulation) for the presence of compounds or compound-bearing supports that have entered the blood or lymph of the test animal. The compounds or compound-bearing supports can be deposited in any body compartment that contains transport proteins capable of transporting a compound or compound-bearing support into a second body compartment, especially the intestinal lumen and the central nervous system compartment.

As with the in vitro methods, the compounds or compound-bearing supports typically include a reporter. The reporter can be a capture tag that facilitates the retrieval and concentration of compounds or compound-bearing supports that are transported. Suitable capture tags, include for example, biotin, magnetic particles associated with the library complex, haptens of high affinity antibodies, and high density metallic particles such as gold or tungsten. The complexes may also include a detection tag to further enhance the retrieval and detection process. As the name implies, detection tags are molecules that are readily identifiable and can be used to monitor the retrieval and concentration of transported compounds or compound-bearing supports. Examples of such molecules include fluorescent molecules, amplifiable DNA molecules, enzymatic markers, and bioactive molecules.

C. Assays for Antimicrobial Activity

The compounds or compound bearing supports of the invention can also be used in screens to identify compounds having antimicrobial activity, i.e., the ability to retard or kill microorganisms (e.g., bacteria, viruses, fungi and parasites). One suitable approach is described in WO 95/12608 (incorporated by reference in its entirety). In brief, cells are plated on agar plates and then overlayed with a layer of agar into which compound-bearing supports are suspended at a suitable dilution so that individual packages can be picked using a capillary for example. The compounds borne by the support are released, such as by cleavage of a linker attached to the compounds. An aliquot of the compounds is reserved for later mass spectral analysis. The agar plate is cultured to allow diffusion of the compounds through the upper layer of agar down to the layer containing cells. The extent to which the released compounds affects the growth or morphology of the cells is monitored. Compounds added to zones showing the desired response (e.g., death) can then be decoded to identify the compound originally attached to the package.

D. Signal Transduction Assays

Cells can be genetically engineered so that upon binding of a compound to a receptor signal transduction triggers the formation of a detectable signal. For example, an exogenous gene encoding an enzyme can be inserted into a site where the exogenous gene is under the transcriptional control of a promoter responsive to a signal transducing receptor. Thus, binding to the receptor triggers the formation of the protein which can react with a substrate within the cell to generate a detectable signal. Using such cells, the compound-bearing supports can be screened for the ability of a pair of compounds borne by the support to bind a receptor and transduce a signal within the cell. Related assays can be conducted to identify compounds capable of agonizing or antagonizing a signal transducing receptor. (See, e.g., U.S. Pat. Nos. 5,401,629 and 5,436,128, which are incorporated by reference in their entirety for all purposes).

V. Decoding

The next step following the identification of a compound that has a desired property is to determine its chemical composition, i.e., to determine the different components that form the compound. A decoding step common to all the methods is to cleave the compounds from the support and subject the cleaved compounds to mass analysis to determine the molecular weight of the compounds borne by the support which bears an active compound. Typically, the molecular weight determination is done by mass spectrometry. As described above in the general description of the method, the molecular weight difference encodes for the two components added during the mixed coupling cycle. Other components are determined on the basis of the pre-encoding (e.g., detection of label) or spatial encoding strategies discussed above. The techniques used to decode labeled components varies according to the nature of the label. For example, IR chromophores are identified by IR spectroscopy. Similarly, NMR active nuclei are detected using NMR spectroscopy, and fluorophores are detected using fluorometers. If all the components are not encoded using one of these techniques, then the remaining component is identified by subtracting the total molecular weight of all the components except the unknown component from the molecular weight of the compound. This difference is equivalent to the molecular weight of the unknown component and thus can be used to identify the unknown component.

The compound pair(s) so identified are then separately resynthesized and then separately assayed to determine which compound is the active compound, whether both are active or whether the observed activity is dependent upon the presence of both compounds. As described above, by judiciously selecting the members of the component pair, it is possible to control to some extent whether the observed activity is more (or less) likely to be a consequence of the cumulative activity of the compounds borne by the support.

VIII. Options Subsequent to Screening

A. Modification of Lead Compound

Once a compound or multiple compounds have been identified after an initial round of screening as having a desired characteristic or activity (a lead compound or lead compounds), the compound(s) can serve as the basis for additional rounds of screening tests. For example, if several different compounds are identified in an initial round, the compounds can be analyzed for common structural features or functionality. Based upon such common features, another library incorporating one or more of the common features or functionalities can be synthesized and subjected to another round of screening to identify compounds that are potentially more active than the compounds identified initially. Alternatively, a new set of compounds derived from each of the positive compounds identified in the initial screening can be synthesized and utilized in another round of screening. This process can be repeated in an iterative manner until the desired degree of refinement in the compound is obtained.

B. Formulation of Active Compounds into Pharmaceutical Compositions

Compounds identified through the screening and rescreening processes described above to have a desired biological activity can be incorporated into pharmaceutical compositions. Typically, such compounds are combined with pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's Pharmaceutical Sciences, Mace Publishing Company, Philadelphia, Pa., 17th ed. (1985); for a brief review of methods for drug delivery, see, Langer, Science 249:1527-1533 (1990), both of which are incorporated by reference in its entirety.

The compositions can be administered for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, compositions are administered to a patient susceptible to or otherwise at risk of a particular disease or infection. Hence, a “prophylactically effective” is an amount sufficient to prevent, hinder or retard a disease state or its symptoms. In either instance, the precise amount of compound contained in the composition depends on the patient's state of health and weight.

An appropriate dosage of the pharmaceutical composition is readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The pharmaceutical compositions can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. The route of administration depends in part on the chemical composition of the active compound and any carriers.

Particularly when the compositions are to be used in vivo, the components used to formulate the pharmaceutical compositions of the present invention are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The following examples are provided to illustrate certain aspects of the invention and are not to be construed to limit the invention.

Unless otherwise stated, all temperatures are in degrees Celsius. Also, in these examples as well as in FIGS. 1-10, unless otherwise defined below, the abbreviations employed have their generally accepted meanings:

Alloc=Allyloxycarbonyl

Boc=Butoxycarbonyl

DIEA=diisopropylethylamine

DMA=5-(N,N-Dimethyl)amiloride Hydrochloride

DMAP=4-Dimethylaminopyridine

DMF=N,N,-Dimethylformamide

Fmoc=9-fluorenyl-methoxycarbonyl

g=gram

h=hour(s)

HATU=O-(7-Azabenzotriazol-1-yl)-N,N,N′N′-tetramethyluronium hexafluorophosphate

kDa=kilo Dalton

LC-MS=liquid chromatography—mass spectrometry

M=molar

mg=milligram

mL=milliliter

min=minute(s)

mM=millimolar

mmole=millimole

Phg=phenylglycine

Pmc=2,2,5,7,8-pentamethylchromane-6-sulfoxyl

TFA=trifluoroacetic acid

THF=tetrahydrofuran

Trt=trityl

(v/v)=volume to volume

(v:v)=volume:volume

μm=micrometer

μL=micro liter

### EXAMPLE 1

### Synthesis of 4000-Member Tripeptide Library Using Orthogonal Protecting Group Chemistry

As outlined in FIG. 3, 10×1 g aliquots of Bromoethyl Photolinker AM resin (100-200 Mesh, loading 1 mmole/g, Novabiochem) are each treated with 50 mL of a DMA solution containing 200 mM Cs₂CO₃ and 5 mmoles of an Alloc-protected amino acid and 5 mmoles of the same Fmoc-amino acid, where the amino acids are one of Gly, Ala, Pro, Val, Leu, Asn, Gln, Met, Phg and Phe (available from Novabiochem). The resins are agitated for 2 h then thoroughly washed with DMF (3×) and CH₂Cl₂ then dried in vacuo. Each aliquot is then treated with 5 mL of a 20% (v/v) solution of piperidine in DMF for 20 min to remove the Fmoc protecting groups. The resins are then thoroughly washed with DMF (3×) and CH₂Cl₂ then dried in vacuo.

Fmoc amino acids are then coupled for 4 h to the resins using HATU as the coupling agent in 25 mL of DMF, the reactions containing 200 mM amino acid, 200 mM HATU and 400 mM DIEA. The 1st aliquot receives Fmoc-Met, the 2^{nd} receives FmocGlu(O^{t}Bu), the 3^{rd} receives Fmoc-His(Boc), the 4^{th} receives Fmoc-Lys(Boc), the 5^{th} receives Fmoc-Arg(Pmc), the 6^{th} receives Fmoc-Phe, the 7^{th} receives Fmoc-Tyr(O^{t}Bu), the 8^{th} receives Fmoc-Gln, the 9^{th} receives Fmoc-Asp(O^{t}Bu) and the 10^{th} receives Fmoc-Trp(Boc). The resins are then thoroughly washed with DMF (3×) and CH₂Cl₂ then dried in vacuo. The Alloc protecting groups are removed by addition of a solution containing Pd(PPh₃)₄ (0.2 mmol), tetrabutylammonium fluoride (3 mmol) and Me₃SiN₃ (8 mmol) in a CH₂Cl₂ (20 mL), and after 30 min agitation under a nitrogen atmosphere, the resins are drained then washed with CH₂Cl₂ (3×). Fmoc amino acids are then coupled for 4 h to the freshly liberated amines using HATU as the coupling agent in 25 mL of DMF, the reactions containing 200 mM amino acid, 200 mM HATU and 400 mM DIEA. The 1st aliquot receives Fmoc-Cys(Trt), the 2^{nd} receives Fmoc-Pro the 3^{rd} receives Fmoc-Thr(O^{t}Bu), the 4^{th} receives Fmoc-Ser(O^{t}Bu), the 5^{th} receives Fmoc-Leu, the 6^{th} receives Fmoc-Val, the 7^{th} receives Fmoc-Ile, the 8^{th} receives Fmoc-Ala, the 9^{th} receives Fmoc-Gly and the 10^{th} receives Fmoc-Asn. The resins are then thoroughly washed with DMF (3×) and CH₂Cl₂ then dried in vacuo.

The resins are next pooled, thoroughly mixed and then split into 20 equal sized aliquots. The Fmoc protecting groups are removed from each aliquot by addition of 2.5 mL of a 20% (v/v) solution of piperidine in DMF for 20 min, and the resins then thoroughly washed with DMF (3×) and CH₂Cl₂ then dried in vacuo. One of 20 different Fmoc amino acids are then coupled for 4 h to the resins using HATU as the coupling agent in 10 mL of DMF, the reactions containing 200 mM amino acid, 200 mM HATU and 400 mM DIEA.

The resins are then thoroughly washed with DMF (3×) and CH₂Cl₂. The Fmoc protecting groups are removed from each aliquot by addition of 2.5 mL of a 20% (v/v) solution of piperidine in DMF for 20 min, and the resins then thoroughly washed with DMF (3×) and CH₂Cl₂ then dried in vacuo. The acid labile side-chain protecting groups are removed from each aliquot by addition of 2.5 mL of a 90:5:5 solution of TFA: H₂O: Et₃SiH. After agitation for 30 min, the resins are drained, washed with CH₂Cl₂ (3×) and then dried in vacuo. Single resin particles can then be selected with a micromanipulator, placed in clean glass micro vials (National Scientific part #C-4008-632C) with ⁱPrOH (5 μL) and photolyzed with 365 nm radiation for 1 h to generate a sample for analysis by flow injection LC-MS analysis using an HP-1100 LC/MSD Engine.

### EXAMPLE 2

### Synthesis of 4000-Member Tripeptide Library Using “Isokinetic” Monomer Mixture Coupling

As summarized in FIG. 4, 10×1 g aliquots of Hydroxymethyl Photolinker AM resin (100-200 Mesh, loading 1 mmole/g, Novabiochem) are coupled with one of 10 Fmoc amino acids (Gly, Ala, Pro, Val, Leu, Asn, Gln, Met, Phg and Phe from Novabiochem) using HATU as the coupling agent in 25 mL of DMF, the reactions containing 200 mM amino acid, 200 mM HATU and 400 mM DIEA. The aliquots are agitated for 4 h then thoroughly washed with DMF (3×) and CH₂Cl₂ then dried in vacuo. The resin is pooled and treated with 50 mL of a 20% (v/v) solution of piperidine in DMF for 20 min to remove the Fmoc protecting groups then thoroughly washed with DMF (3×) and CH₂Cl₂ and then dried in vacuo.

The resin is divided into 10 equal aliquots and coupled with equimolar mixture of 2 Fmoc amino acids for 4 h using HATU as the coupling agent in 50 mL of DMF, the reactions containing 200 mM amino acid, 200 mM HATU and 400 mM DIEA. The 1st aliquot receives Fmoc-Ile and Fmoc-Thr(O^{t}Bu), the 2^{nd} receives Fmoc-Lys(Boc) and FmocAsp(O^{t}Bu), the 3^{rd} receives Fmoc-Ala and Fmoc-Gly, the 4^{th} receives Fmoc-Asn and FmocVal, the 5^{th} receives Fmoc-Cys(Trt) and Fmoc-Ser(O^{t}Bu), the 6^{th} receives Fmoc-His(Boc) and Fmoc-Glu(O^{t}Bu), the 7^{th} receives Fmoc-Trp(Boc) and Fmoc-Tyr(O^{t}Bu), the 8^{th} receives Fmoc-Arg(Pmc) and Fmoc-Gln, the 9^{th} receives Fmoc-Phe and Fmoc-Leu and the 10^{th} receives Fmoc-Met and Fmoc-Pro. The resins are then thoroughly washed with DMF (3×) and CH₂Cl₂ then dried in vacuo.

The resins are next pooled, thoroughly mixed and then split into 20 equal sized aliquots. The Fmoc protecting groups are removed from each aliquot by addition of 2.5 mL of a 20% (v/v) solution of piperidine in DMF for 20 min, and the resins then thoroughly washed with DMF (3×) and CH₂Cl₂ then dried in vacuo. One of 20 different Fmoc amino acids are then coupled for 4 h to the resins using HATU as the coupling agent in 10 mL of DMF, the reactions containing 200 mM amino acid, 200 mM HATU and 400 mM DIEA. The resins are then thoroughly washed with DMF (3×) and CH₂Cl₂. The Fmoc protecting groups are removed from each aliquot by addition of 2.5 mL of a 20% (v/v) solution of piperidine in DMF for 20 min, and the resins then thoroughly washed with DMF (3×) and CH₂Cl₂ then dried in vacuo.

The acid labile side-chain protecting groups are removed from each aliquot by addition of 2.5 mL of a 90:5:5 solution of TFA: H₂O: Et₃SiH. After agitation for 30 min, the resins are drained, washed with CH₂Cl₂ (3×) and then dried in vacuo. Single resin particles can then be selected with a micromanipulator, placed in clean glass micro vials (National Scientific part #C-4008-632C) with ⁱPrOH (5 μL) and photolyzed with 365 nm radiation for 1 h to generate a sample for analysis by flow injection LC-MS analysis using an HP-1100 LC/MSD Engine.

### EXAMPLE 3

### Synthesis of a 4096-Member N-Acyl-N-Alkyl Amino Acid Amide Library with Fluorescent Microbead Pre-Encoding

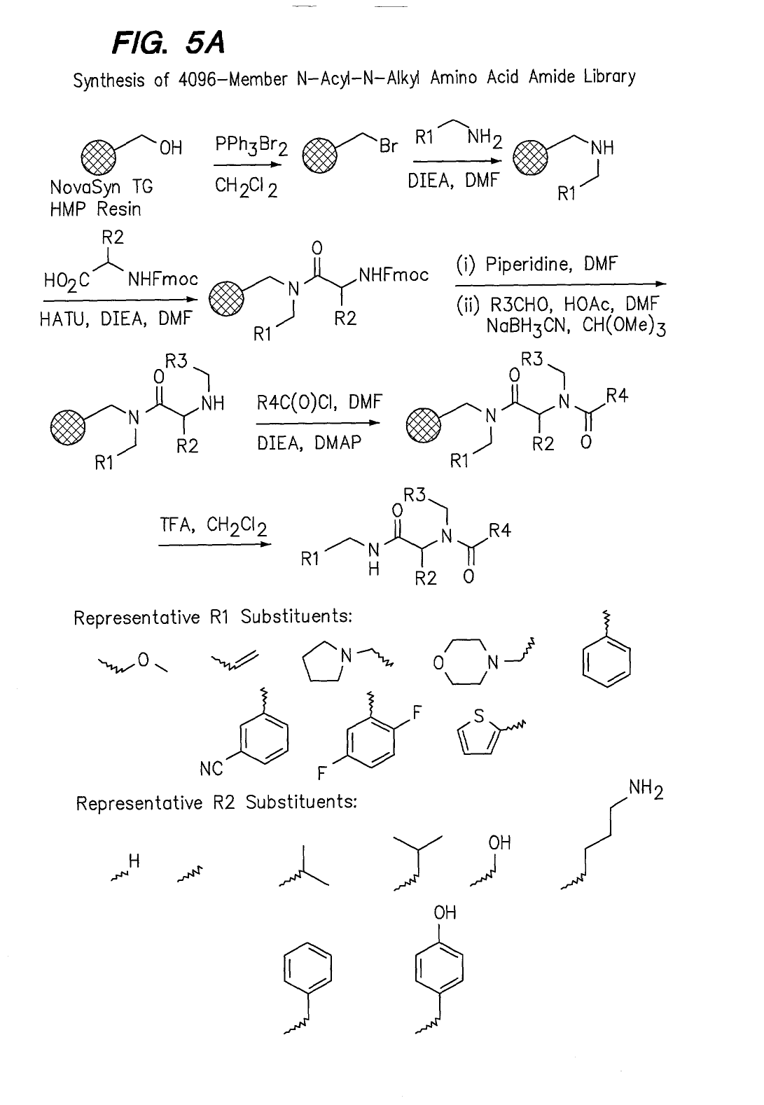

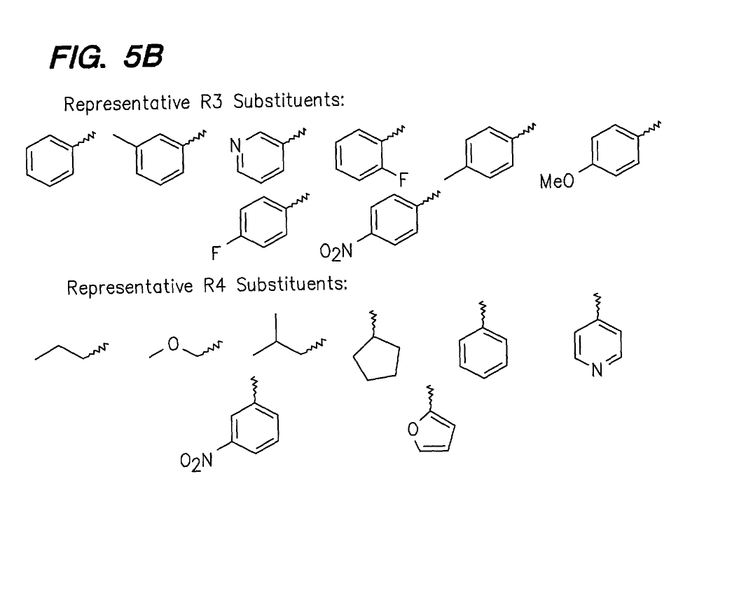

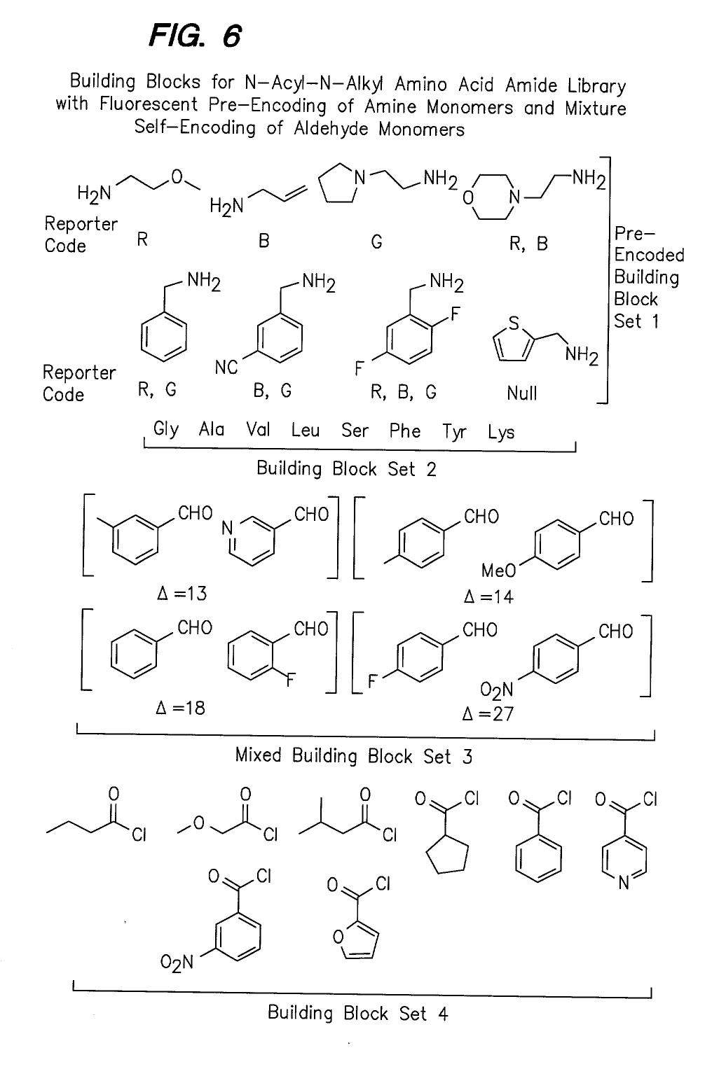

As represented in FIG. 5, 10 g NovaSyn TG HMP resin (loading 0.3 mmol/g) is converted to the bromide derivative by treatment with PPh₃Br₂ (3 mmole) in for CH₂Cl₂ (50 mL) 4 h at room temperature. The resin is drained and washed thoroughly with CH₂Cl₂ (3×) and then dried in vacuo. The resin is then partitioned into 8 equal sized aliquots and reacted with 50 mL of a DMF solution containing 1 M DIEA and 2.5 mmole of one of 8 different primary amines from the Building Block Set 1 (FIG. 6). After agitation for 12 h, the resin is thoroughly washed with DMF and CH₂Cl₂ then dried in vacuo. 500 mg of each resin aliquot is then removed and separately encoded by non-covalent fluorescent labeling according to the method of Trau (PCT Application WO 99/24458). Briefly, 1 μm diameter fluorescent silica particles (red, green and blue sicastar beads, obtained from MicroMod Particle Technologies, GmbH) are coated with polyelectrolyte overlayers by overnight treatment with a 1% aqueous solution of 10 kDa polyethyleneimine, washing and then overnight treatment with a 1% aqueous solution of 250 kDa polyacrylic acid. The eight possible binary combinations of reporter beads (i.e. the combinations red; blue; green; red and blue; red and green; blue and green; red and blue and green; null) are prepared by mixing suspensions of the beads in DMF at 5 mg beads/mL. Each 500 mg resin aliquot is treated with 20 mL of these 8 reporter bead combinations for 5 min according to the labeling scheme in FIG. 6. Multiple reporters become non-covalently attached to every resin particle and the remaining reporters are washed away completely with DMF.

The labeled resin aliquots are then pooled, thoroughly mixed and split again into 8 equal sized aliquots. Each is then reacted with one of 8 Fmoc-protected amino acids (Fmoc-Gly, Fmoc-Ala, Fmoc-Val, Fmoc-Leu, Fmoc-Ser(O^{t}Bu), Fmoc-Phe, Fmoc-Tyr(O^{t}Bu) and Fmoc-Lys(Boc)) for 4 h using HATU as the coupling agent in 5 mL of DMF, the reactions containing 200 mM amino acid, 200 mM HATU and 400 mM DIEA (see FIGS. 5 and 6). The resins are drained and then thoroughly washed with DMF (3×) and CH₂Cl₂, then dried in vacuo.

The resins are pooled again and the Fmoc protecting groups are removed by addition of 20 mL of a 20% (v/v) solution of piperidine in DMF for 20 min, and the resins then thoroughly washed with DMF (3×) and CH₂Cl₂ then dried in vacuo. The resin is then split into 4 equal sized aliquots and each aliquot is reacted separately under standard reductive alkylkation conditions (see Schwarz et al, (1999) J. Org. Chem. 64: 2219) with a different pair of aldehydes. As outlined in FIG. 6, the 1^{st} aliquot receives m-tolualdehyde and 3-pyridinecarboxaldehyde; the 2^{nd} aliquot receives p-tolualdehyde and 4-methoxybenzaldehyde; the 3^{rd} aliquot receives benzaldehyde and 2-fluorobenzaldehyde; and the 4^{th} aliquot receives 4-fluorobenzaldehyde and 4-nitrobenzaldehyde. These reactions, containing 2 mmole of each aldehyde and 3 mL of a 6% (v/v) solution of HOAc in MeOH dissolved in 20 mL of dry CH(OMe)₃/DMF (9:1), are gently warmed to 40° C. for 12 h before addition of 20 mL of a I M solution of NaBH₃CN in THF. After further agitation for 6 h, the resins are drained and washed thoroughly with MeOH, H₂O, DMF and CH₂Cl₂, then dried in vacuo.

The resins are pooled again and then split into 8 equal sized aliquots for reaction with one of 8 different acyl chlorides shown in FIG. 6. These reactions are performed for 4 h in 5 mL of DMF containing 200 mM acyl chloride, 400 mM DIEA and 20 mM DMAP. The resins are drained and then thoroughly washed with DMF (3×) and CH₂Cl₂, then dried in vacuo. Single resin particles from any pool can then be decoded by selection with a micromanipulator, placed in clean glass micro vials (National Scientific part #C-4008-632C) and treated for 1 h with 100 μL of 50% (v:v) TFA in CH₂Cl₂ to cleave the pair of compounds from the bead. After thorough evaporation of all volatiles in vacuo, the residue is dissolved in 20 μL of MeOH to generate a sample for analysis by flow injection LC-MS analysis using an HP-1100 LC/MSD Engine. The fluorescent reporter beads on the synthesis particle are imaged using a fluorescence microscope (Olympus IX70) equipped with a series of excitation and bandpass filters (ex. 330-385 nm, em.>420 nm; ex 450-480 nm, em >515 nm; ex 510-550 nm, em >590 nm).

### EXAMPLE 4

### Synthesis of a 9216-Member 1,5-Benzodiazepin-5-one Library with Fluorescent Microbead Pre-Encoding

As shown in FIG. 7, 6 g NovaSyn TG HMP resin (loading 0.3 mmol/g) is converted to the bromide derivative by treatment with PPh₃Br₂ (3 mmole) in for CH₂Cl₂ (50 mL) 4 h at room temperature. The resin is drained and washed thoroughly with CH₂Cl₂ (3×) and then dried in vacuo. The resin is then partitioned into 3 equal sized aliquots and reacted with 10 mL of a DMF solution containing 1 M DIEA and 5 mmole of either oiodobenzylamine, m-iodobenzylamine orp-iodobenzylamine. After agitation for 12 h, the resin is thoroughly washed with DMF and CH₂Cl₂ then dried in vacuo. 1 g aliquots of each of these resins are taken and divided into two equal portions. These 6 samples are labeled non-covalently with binary combinations of 1 μm fluorescent reporter microbeads as described in Example 3 above. The following combinations are used: o-iodobenzylamine aliquot 1-red; o-iodobenzylamine aliquot 2-red and green; m-iodobenzylamine aliquot 1-green; m-iodobenzylamine aliquot 2-green and blue; p-iodobenzylamine aliquot 1-blue; p-iodobenzylamine aliquot 2-red and blue.

The first aliquot of each labeled amine sample is treated with 4-fluoro-3-nitrobenzoic acid for 4 h using HATU as the coupling agent in 5 mL of DMF, the reactions containing 200 mM of the benzoic acid, 200 mM HATU and 400 mM DIEA. The resins are drained and then thoroughly washed with DMF (3×) and CH₂Cl₂, then dried in vacuo. The second aliquot of each labeled amine sample is treated with 3-fluoro-4-nitrobeiizoic acid for 4 h using HATU as the coupling agent in 5 mL of DMF, the reactions containing 200 mM of the benzoic acid, 200 mM HATU and 400 mM DIEA. The resins are drained and then thoroughly washed with DMF (3×) and CH₂Cl₂, then dried in vacuo.

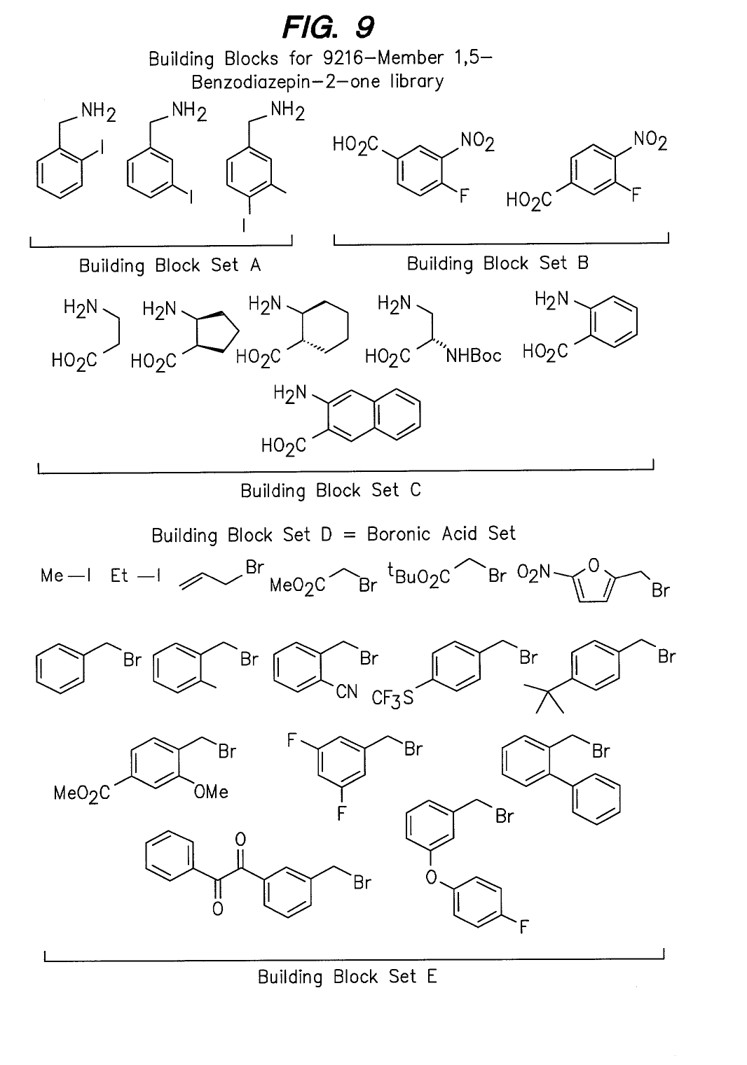

The six samples are then pooled, mixed thoroughly and redivided into 6 aliquots of equal size. Each sample is treated with one of 6 β-amino acids shown in Building Block Set C in FIG. 9, dissolved at 0.2M in acetone/aq. NaHCO₃ (1:1) and the resins agitated at 75° C. for 24 h. Note that the anthranilic acid reactions are allowed to proceed for 72 h rather than 24 h. After draining the resins are washed with 5% aq. HOAc, H₂O, MeOH, DMF and CH₂Cl₂, then dried in vacuo. The resins are pooled, mixed and redivided into 8 equally sized aliquots. Suzuki cross coupling reactions are then preformed using the boronic acid pairings shown in FIG. 8. Each reaction is run for 12 h at 65° C. in 5 mL DMF and contains 0.5 mmole of each of the 2 boronic acids, 0.02 mmole [PdCl₂(dppf)] and 10 mmole NEt₃. The resins are cooled and washed thoroughly with DMF and CH₂Cl₂, then dried in vacuo. The samples are pooled and the aromatic nitro groups reduced by treatment with SnCl₂.2H₂O (100 mmole) in 50 mL DMF at room temperature for 24 h. The resin is drained and washed with DMF, CH₂Cl₂, MeOH and CH₂Cl₂, then dried in vacuo. The benzodiazepinone cyclization is performed by addition of 80 mL of a 200 mM solution of DIEA in DMF followed by 16 mmole of diethyl cyanophosphate. After 8 h the resin is drained and washed extensively with DMF, CH₂Cl₂, MeOH and CH₂Cl₂, then dried in vacuo.

The resin is divided into 16 equal sized aliquots and each was alkylated with one of the 16 alkyl bromides/iodides from Building Block Set E shown in FIG. 9. To each aliquot is added 6 mL of a 2M solution of the alkylating agent in DMF and the reaction allowed to proceed at 55° C. for 3 days. The resin is drained and washed with DMF, CH₂Cl₂, MeOH and CH₂Cl₂, then dried in vacuo.

Single resin particles from any pool can then be decoded by selection with a micromanipulator, placed in clean glass micro vials (National Scientific part #C-4008-632C) and treated for 1 h with 100 μL of 50% (v:v) TFA in CH₂Cl₂ to cleave the pair of compounds from the bead. After thorough evaporation of all volatiles in vacuo, the residue is dissolved in 20 μL of MeOH to generate a sample for analysis by flow injection LC-MS analysis using an HP-1100 LC/MSD Engine. The fluorescent reporter beads on the synthesis particle are imaged using a fluorescence microscope (Olympus IX70) equipped with a series of excitation and bandpass filters (ex. 330-385 nm, em.>420 nm; ex 450-480 nm, em>515 nm; ex 510-550 nm, em>590 nm).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

What is claimed is:

1. A method of screening a library of compounds, comprising:
(a) conducting a plurality of synthesis cycles to synthesize compounds on supports in a component-by-component fashion, a synthesis cycle comprising apportioning supports into reaction vessels and reacting the supports in different vessels with different components of the compounds, whereby the components attach to the supports or with components attached to the supports in previous cycles, and the supports from different vessels are pooled between synthesis cycles;
wherein at least one cycle is conducted by contacting different vessels of supports with different paired components, the members of each pair of components attaching independently to the supports or components attached thereto in a previous cycle, whereby supports in the same vessel receive the same pair of components, and supports in different vessels receive different pairs of components, the components in each pair having a known difference in molecular weight, and the differences in molecular weights varying between pairs, to produce a population of supports bearing different pairs of compounds, the members of the pairs of compounds having a known difference in molecular weight;
(b) assaying the supports bearing different paired compounds, and isolating at least one support wherein at least one of the paired compounds on the isolated support has a desired property; and
(c) performing a determining step comprising determining the molecular weights of each of the compounds of the pair borne by the at least one isolated support, the difference in molecular weight between the members of a pair of compounds indicating which pair of components was incorporated into the pair of compounds in the at least one cycle.

2. The method of claim 1, wherein one of the plurality of synthesis cycles is a synthesis cycle that precedes the at least one cycle, the cycle preceding the at least one cycle comprising:
(d) apportioning the supports into a plurality of first reaction vessels and reacting the supports with different first components in the different reaction vessels, whereby the first components attach to the support; and
(e) labeling the supports by reacting the supports in the plurality of first reaction vessels with different labels, such that supports within a reaction vessel bear the same label, but supports within different reaction vessels bear different labels; and
wherein the determining step further comprises determining which label is associated with the at least one isolated support, the label indicating which first component was added during the cycle preceding the at least one cycle.

3. The method of claim 1, wherein one of the plurality of synthesis cycles is a synthesis cycle that precedes the at least one cycle, the cycle preceding the at least one cycle comprising:
(d) providing a collection of supports comprising different labels, there being a plurality of supports bearing each label; and
(e) apportioning the supports into a plurality of first reaction vessels, such that each reaction vessel contains supports bearing the same label, but supports in different reaction vessels bear different labels, and reacting the labeled supports with different first components in the different reaction vessels, whereby the first components attach to the labeled support; and
wherein the determining step further comprises determining which label is associated with the at least one isolated support, the label indicating which first component was added during the cycle preceding the at least one cycle.

4. The method of claim 3, wherein the label comprises a physical characteristic of the support.

5. The method of claim 4, wherein the physical characteristic is selected from the group consisting of the shape of the support, the size of the support and an alphanumeric tag formed into the support.

6. The method of claim 3, wherein the label is selected from the group consisting of a fluorescent label, a chromophore, a radiolabel, a magnetic particle, an electron dense particle, an infra-red chromophore, an NMR active nuclei and a fluorescent microbead.

7. The method of claim 6, wherein the determining step comprises determining the identity of the label by confocal microscopy, semi-confocal microscopy, infra-red spectroscopy, NMR spectroscopy or by detecting the label with a charge-coupled device.

8. The method of claim 1, wherein the plurality of synthesis cycles comprises a plurality of synthesis cycles preceding the at least one cycle, the plurality of synthesis cycles preceding the at least one cycle, comprising:
(d) in a first synthesis cycle, apportioning the supports into a plurality of first reaction vessels and reacting the supports with different first components in the different vessels, the first components attaching to the supports;
(e) in a second synthesis cycle,
(i) splitting the supports from each of the plurality of first reaction vessels into a set of multiple reaction vessels, the sets forming a plurality of second reaction vessels;
(ii) labeling the supports in each of the second reaction vessels with a different label, such that supports in a reaction vessel have the same label, but supports in different reaction vessels have different labels; and
(iii) reacting the supports in different reaction vessels of each set with different second components, whereby the second component attaches to the support via the first component; and
wherein the determining step further comprises determining the identity of the label associated with the at least one isolated support, the label indicating which components were added during the first and second synthesis cycles.

9. The method of claim 8, wherein step (iii) is performed before step (ii).

10. The method of claim 1, wherein the plurality of synthesis cycles comprises a plurality of synthesis cycles preceding the at least one cycle, the plurality of synthesis cycles preceding the at least one cycle, comprising:
(d) in a first synthesis cycle, apportioning the supports into a plurality of first reaction vessels and reacting the supports with different first components in the different vessels, the number of different vessels to which any particular first component is added being equal to the number of different second components added in a second synthesis cycle, and whereby the first components attach to the supports;
(e) in the second synthesis cycle, reacting supports in the plurality of first reaction vessels with different second components, wherein supports in different first reaction vessels that were reacted with the same first component during the first synthesis cycle are reacted with different second components, whereby the second components attach to the support via the first component; and
wherein the determining step further comprises determining the identity of the label associated with the at least one isolated support, the label indicating which components were added during the first and second synthesis cycles.

11. The method of claim 1, wherein the plurality of synthesis cycles comprises a final synthesis cycle performed subsequent to the at least one cycle, the final synthesis cycle comprising:
(d) apportioning the supports into a plurality of final reaction vessels, and reacting supports with different final components in the different reaction vessels, whereby the components attach to the support via the components added in one of the preceding synthesis cycles, and wherein the identity of each final component in each final reaction vessel is tracked such that the identity of the component added to each of the final reaction vessels is known;
and wherein:
the assaying step comprises separately removing supports and assaying the supports bearing the paired compounds from each of the final reaction vessels; and
the determining step further comprises identifying the component added during the final synthesis step on the basis of which reaction vessel the at least one support was obtained from for the assaying step.

12. The method of claim 1, wherein one of the plurality of synthesis cycles is a synthesis cycle performed subsequent to the at least one cycle, the cycle subsequent to the at least one cycle comprising:
(d) apportioning the supports into a plurality of second reaction vessels and contacting different vessels of supports with different second paired components, the members of each second pair attaching independently to the support via a component added in one of the preceding steps, whereby supports in the same vessel receive the same pair of components, and supports in different vessels receive different pairs of components, the components in each second pair having a known difference in molecular weight, and the differences in molecular weights varying between second pairs, to produce a population of supports further bearing a second pair of compounds, the members of the second pair having a known difference in molecular weight; and
wherein the determining step further comprises determining the molecular weights of each of the compounds of the second pair borne by the at least one isolated support, the difference in molecular weight between the members of the second pair of compounds indicating which pair of components was incorporated into the compounds in the cycle subsequent to the at least one cycle.

13. The method of claim 1, wherein the contacting step comprises adding paired components so that the members of each pair of components attach to the support at approximately equal rates, whereby the relative concentrations of the members of the pair of compounds borne by the at least one isolated support is substantially the same.

14. The method of claim 13, wherein the regulating step comprises adding a lesser amount of the more reactive member of each of the paired components to the supports.

15. The method of claim 13, wherein the members of the paired components are electronically similar.

16. The method of claim 13, wherein the members of the paired components are sterically similar.

17. The method of claim 1, wherein the members of each component pair are electronically and/or sterically dissimilar such that the members of each compound pair differ in reactivity as to a selected biological activity.

18. The method of claim 1, wherein the members of each component pair are electronically and/or sterically similar such that the members of each compound pair have similar reactivity as to a selected biological activity.

19. The method of claim 1, wherein the components are selected from groups consisting of amino acids, carbohydrates, lipids, phospholipids, carbamates, sulfones, sulfoxides, esters, nucleosides, amines, carboxylic acids, aldehydes, ketones, isocyanates, isothiocyanates, thiols, alkyl halides, phenolic molecules, boronic acids, stannanes, alkyl or aryl lithium molecules, Grignard reagents, alkenes, alkynes, dienes, ureas and other heterocyclic molecules.

20. The method of claim 1, wherein the compounds are selected from the group consisting of a polypeptide, an oligosaccharide, an oligonucleotide, a phospholipid, a lipid, a benzodiazepine, a thiazolidinone, an imidizolidinone and other heterocyclic molecules.

21. The method of claim 1, wherein the supports are selected from the group consisting of a nanoparticle and a molecular scaffold.

22. The method of claim 1, wherein the supports are selected from the group consisting of a glass bead, a latex bead, a polystyrene bead and a metal particle.

23. The method of claim 1, wherein the supports include a linker to which components can be attached.

24. The method of claim 23, wherein the linker is cleavable and the assaying step comprises cleaving the linkers.

25. The method of claim 1, wherein the desired property is selected from the group consisting of the capacity to bind a receptor, the capacity to be transported into or through a cell, the capacity to be a substrate or inhibitor for an enzyme, the capacity to kill bacteria, the capacity to agonize or antagonize a receptor.

26. The method of claim 1, wherein the desired property is selected from the group consisting of a catalytic activity, conductivity, resistivity, and a dielectric property.

27. The method of claim 25, wherein the desired property is capacity to bind a receptor, and the assaying step comprises cleaving the paired compounds from the supports, contacting the compounds with the receptor and identifying at least one compound that binds to the receptor.

28. The method of claim 25, wherein the desired property is the capacity to be transported into or through a cell and the assaying step comprises contacting the supports bearing different paired compounds with the cell and identifying at least one support bearing a compound that is transported into or through the cell.

29. The method of claim 28, wherein the contacting step of the assaying step is performed in vitro.

30. The method of claim 28, wherein the contacting step of the assaying step is performed in vivo.

31. The method of claim 30, wherein:
(a) the contacting step of the assaying step comprises introducing supports bearing different compounds into a body compartment or tissue of an animal;
(b) the cell is a plurality of cells in the body compartment or tissue; and
(c) the identifying step comprises retrieving the at least one support bearing paired components from a tissue or fluid after transport of the at least one support through the cells lining the cavity.

32. The method of claim 1, wherein the determining step comprises determining the molecular weight of the paired components borne by the at least one isolated support by mass spectroscopy.

33. The method of claim 1, further comprising:
(d) synthesizing the members of the pairs of compounds borne by the at least one isolated support; and
(e) separately assaying the synthesized compounds for the desired property.

34. The method of claim 1, wherein the assaying step comprises detecting a reporter attached to the support.

35. The method of claim 34, wherein the reporter is a label selected from the group consisting of a fluorophore, a chromophore, a radiolabel, a magnetic particle, an electron dense particle and a substrate for an enzyme.

36. A method of screening a library of compounds, comprising:
(a) in a first synthesis cycle, apportioning a collection of labeled supports comprising different labels into a plurality of first reaction vessels so that the labeled supports in a reaction vessel are the same, but the labeled supports in different reaction vessels are different; and reacting the supports with different first components in the different first vessels, whereby the first components attach to the support either directly or optionally via some linker or spacer component;
(b) in a second synthesis cycle, pooling the supports, and apportioning the supports in a second plurality of reaction vessels, and reacting the supports with different paired components, the members of each pair having a known difference in molecular weight, the difference in molecular weight differing between pairs, whereby the members of each pair attach independently to the support via a component added in a preceding step;
(c) in a third synthesis cycle, pooling the supports and apportioning the supports in a third plurality of reaction vessels, and reacting supports with different third components in the different reaction vessels, whereby the third components attach to the support via a component added in a preceding step;
thereby forming a population of supports, each support bearing different pairs of compounds, the members of the pairs of compounds having a known difference in molecular weight;
(d) assaying the supports bearing different paired compounds, and isolating at least one support wherein at least one of the paired compounds on the isolated support has a desired property; and
(e) determining the molecular weights of each of the paired compounds borne by the at least one isolated support, the difference in molecular weight between the pair of compounds indicating which pair of components was incorporated into the pair of compounds in the second synthesis cycle, the labeling indicating which component was added during the first synthesis cycle, and the total molecular weight of each compound, and the identity of the components added during the first and second synthesis cycles, indicating which component was added during the third synthesis cycle.

37. The method of claim 36, wherein the third components each have different molecular weights.

38. A method of screening a library of compounds, comprising:
(a) apportioning a plurality of supports into a plurality of first reaction vessels;
(b) in a first synthesis cycle, reacting the supports with different first components in the different first vessels, whereby the first components attach to the support;
(c) labeling the supports by reacting the supports in the different first reaction vessels with different labels, such that supports within a reaction vessel bear the same label, but supports within different reaction vessels bear different labels;
(d) in a second synthesis cycle, pooling the supports, and apportioning the supports in a second plurality of reaction vessels, and reacting the supports with different paired components, the members of each pair having a known difference in molecular weight, the difference in molecular weight differing between pairs, whereby the members of each pair attach independently to the support via a component added in a preceding step;
(e) in a third synthesis cycle, pooling the supports and apportioning the supports in a third plurality of reaction vessels, and reacting supports with different third components in the different vessels, whereby the third components attach to the support via a component added in a preceding step;
thereby forming a population of supports, each support bearing different first and second pairs of compounds, the members of the pairs of compounds having a known difference in molecular weight;
(f) assaying the supports bearing different paired compounds, and isolating at least one support wherein at least one of the paired compounds on the isolated support has a desired property; and
(g) determining the molecular weights of each of the paired compounds borne by the at least one isolated support, the difference in molecular weight between the pair of compounds indicating which pair of components was incorporated into the pair of compounds in the second synthesis cycle, the labeling indicating which component was added during the first synthesis cycle, and the total molecular weight of each compound, and the identity of the components added during the first and second synthesis cycles, indicating which component was added during the third synthesis cycle.

39. The method of claim 38, wherein step (c) is performed before step (b).

40. The method of claim 38, wherein step (e) is performed before step (d).

41. The method of claim 38, wherein the third components each have different molecular weights.

42. A method of screening a library of compounds, comprising:
(a) in a first synthesis cycle, apportioning a plurality of supports into a plurality of first reaction vessels; and reacting the supports with different first components in the different vessels, whereby the first components attach to the support or to a component added in a previous step;
(b) in a second synthesis cycle, pooling the supports, and apportioning the supports into a plurality of second reaction vessels, and reacting the supports with different paired components, the members of each pair having a known difference in molecular weight, the difference in molecular weight differing between pairs, whereby the members of each pair attach independently to the support via a component added in a preceding step;
(c) in a third synthesis cycle, pooling the supports and apportioning the supports in a third plurality of reaction vessels, and reacting supports with different third components, whereby the third components attach to the support via a component added in a preceding step, and wherein the identity of each component in each reaction vessel is tracked such that the identity of the third component in each of the third reaction vessels is known;
thereby forming a population of supports, each support bearing different pairs of compounds, the members of the pairs of compounds having a known difference in molecular weight;
(d) separately assaying the supports bearing the paired compounds from each of the plurality of third reaction vessels, and isolating at least one support wherein at least one of the paired compounds on the isolated support has a desired property; and
(e) determining the molecular weights of each of the paired compounds borne by the at least one isolated support, the difference in molecular weight between the pair of compounds indicating which pair of components was incorporated into the pair of compounds in the second synthesis cycle, the identity of the third reaction vessel from which the support was obtained for the assaying step indicating which component was added during the third synthesis cycle, and the total molecular weight of each compound, and the identity of the components added during the second and third synthesis cycles, indicating which component was added during the first synthesis cycle.

43. The method of claim 42, wherein step (b) is performed before step (a).

44. The method of claim 42, wherein the first components each have different molecular weights.

45. A method of screening a library of compounds, comprising:
(a) in a first synthesis cycle, apportioning a collection of labeled supports comprising different labels into a plurality of first reaction vessels so that the labeled supports in a reaction vessel are the same, but the labeled supports in different reaction vessels are different; and reacting the supports with different first components in the different first vessels, whereby the first components attach to the support;
(b) in a second synthesis cycle, pooling the supports, and apportioning the supports in a plurality of second reaction vessels, and reacting the supports with different paired components, the members of each pair having a known difference in molecular weight, the difference in molecular weight differing between pairs, whereby the members of each pair attach independently to the support via a component added in the preceding step;
(c) in a third synthesis cycle, pooling the supports and apportioning the supports in a plurality of third reaction vessels, and reacting the supports with different third components, whereby the third components attach to the support via a component added in a preceding step;
(d) in a fourth synthesis cycle, pooling the supports and apportioning the supports in a plurality of fourth reaction vessels, and reacting supports with different components, whereby the components attach to the support via a component added in the preceding step; and wherein the identity of each fourth component in each reaction vessel is tracked such that the identity of the fourth component added to each of the fourth reaction vessels is known;
thereby forming a population of supports, each support bearing different pairs of compounds, the members of the pairs of compounds having a known difference in molecular weight;
(e) separately assaying the supports bearing the paired compounds from each of the plurality of fourth reaction vessels, and isolating at least one support wherein at least one of the paired compounds on the isolated support has a desired property; and
(f) determining the molecular weights of each of the paired compounds borne by the at least one isolated support, the difference in molecular weight between the pair of compounds indicating which pair of components was incorporated into the pair of compounds in the second synthesis cycle, the labeling indicating which component was added during the first synthesis cycle, the identity of the reaction vessel from which the support was obtained for the assaying step indicating which component was added during the fourth synthesis cycle and the total molecular weight of each compound, and the identity of the components added during the first, second and fourth synthesis cycles, indicating which component was added during the third synthesis cycle.

46. The method of claim 45, wherein step (c) is performed before step (b).

47. The method of claim 45, wherein the third components each have different molecular weights.

48. A method of screening a library of compounds, comprising:
(a) apportioning a plurality of supports into a plurality of first reaction vessels;
(b) in a first synthesis cycle, reacting the supports with different first components in the different vessels, whereby the first components attach to the support;
(c) reacting the supports with different labels in the different reaction vessels, such that supports within a reaction vessel bear the same label, but supports within different reaction vessels bear different labels;
(d) in a second synthesis cycle, pooling the supports, and apportioning the supports in a plurality of second reaction vessels, and reacting the supports with different paired components, the members of each pair having a known difference in molecular weight, the difference in molecular weight differing between pairs, whereby the members of each pair attach independently to the support via a component added in a preceding step;
(e) in a third synthesis cycle, pooling the supports and apportioning the supports in a plurality of third reaction vessels, and reacting the supports with different third components in the different reaction vessels, whereby the components attach to the support via a component added in a preceding cycle;
(f) in a fourth synthesis cycle, pooling the supports and apportioning the supports in a plurality of fourth reaction vessels, and reacting supports with different components, whereby the components attach to the support via a component added in a preceding step, and wherein the identity of each component in each reaction vessel is tracked such that the identity of the fourth component added to each fourth reaction vessels is known;
thereby forming a population of supports, each support bearing different pairs of compounds, the members of the pairs of compounds having a known difference in molecular weight
(g) separately assaying the supports bearing the paired compounds from each of the plurality of fourth reaction vessels, and isolating at least one support wherein at least one of the paired compounds on the isolated support has a desired property; and
(h) determining the molecular weights of each of the pair of compounds borne by the at least one isolated support, the difference in molecular weight between the pair of compounds indicating which pair of components was incorporated into the pair of compounds in the second synthesis cycle, the labeling indicating which component was added in the first synthesis cycle, the identity of the reaction vessel from which the support was obtained for the assaying step indicating which component was added during the fourth synthesis cycle, and the total molecular weight of each compound, and the identity of the components added during the first, second and fourth synthesis cycles, indicating which component was added during the third synthesis cycle.

49. The method of claim 48, wherein step (c) is performed before step (b).

50. The method of claim 48, wherein step (e) is performed before step (d).

51. The method of claim 48, wherein the third components each have different molecular weights.

52. A method of screening a library of compounds, comprising:
(a) in a first synthesis cycle, apportioning a plurality of supports into a plurality of first reaction vessels, and reacting the supports with different first components in the different vessels, whereby the first components attach to the support or to a component added in a preceding step;
(b) in a second synthesis cycle, pooling said supports and apportioning the supports in a plurality of second reaction vessels, and reacting the supports with a first set of different paired components, the members of each pair having a known difference in molecular weight, the difference in molecular weight differing between pairs, whereby the members of each pair attach independently to the support or to the support via a component added in a preceding step;
(c) in a third synthesis cycle, pooling the supports and apportioning the supports in a plurality of third reaction vessels, and reacting the supports with a second set of different paired components, the members of each second pair having a known difference in molecular weight, the difference in molecular weight differing between the second pairs, whereby the members of each second pair attach independently to the support or to the support via a component added in a preceding step;
(d) in a fourth synthesis cycle, pooling the supports and apportioning the supports in a plurality of fourth reaction vessels, and reacting supports with different components, whereby the components attach to the support via a component added in a preceding step, and wherein the identity of each fourth component in each reaction vessel is tracked such that the identity of the fourth component added to each of the fourth reaction vessels is known;
thereby forming a population of supports, each support bearing four different compounds;
(e) separately assaying the supports bearing the four compounds from each of the fourth plurality of reaction vessels, and isolating at least one support wherein at least one of the four compounds on the isolated support has a desired property; and
(f) determining the molecular weights of the four compounds borne by the at least one isolated support, the difference in molecular weight between the members of a first pair of compounds from the four compounds indicating which pair of components was incorporated into the pair of compounds in the second synthesis cycle, the difference in molecular weight between the members of a second pair of compounds from the four compounds indicating which pair of components was incorporated into the pair of compounds in the third synthesis cycle, the location from which the support was obtained in the fourth synthesis cycle indicating which component was added during the fourth synthesis cycle, and the total molecular weight of each compound, and the identity of the components added during the second, third and fourth synthesis cycles, indicating which component was added during the first synthesis cycle.

53. The method of claim 52, wherein steps (a)-(c) are performed in the order (b), (a), (c).

54. The method of claim 52, wherein steps (a)-(c) are performed in the order (b), (c), (a).

55. The method of claim 52, wherein the first components each have different molecular weights.

56. A method of screening a library of compounds, comprising:
(a) apportioning a plurality of supports into a plurality of first reaction vessels;
(b) in a in a first synthesis cycle, reacting the supports with different first components in the different vessels, whereby the first components attach to the support;
(c) reacting the supports with different labels in the different reaction vessels, such that supports within a reaction vessel bear the same label, but supports within different reaction vessels bear different labels;
(d) in a second synthesis cycle, pooling said supports and apportioning the supports in a plurality of second reaction vessels, and reacting the supports with a first set of different paired components, the members of each pair having a known difference in molecular weight, the difference in molecular weight differing between pairs, whereby the members of each pair attach independently to the support or to the support via a component added in a preceding step;
(e) in a third synthesis cycle, pooling the supports and apportioning the supports in a plurality of third reaction vessels, and reacting the supports with a second set of different paired components, the members of each second pair having a known difference in molecular weight, the difference in molecular weight differing between the second pairs, whereby the members of each second pair attach independently to the support or to the support via a component added in a preceding step;
(f) in a fourth synthesis cycle, pooling the supports and apportioning the supports in a plurality of fourth reaction vessels, and reacting the supports with different components, whereby the components attach to the support via a component added in a preceding step;
thereby forming a population of supports, each support bearing four different compounds;
(g) assaying the supports bearing the four compounds from each of the fourth plurality of reaction vessels, and isolating at least one support wherein at least one of the four compounds on the isolated support has a desired property; and
(h) determining the molecular weights of the four compounds borne by the at least one isolated support, the difference in molecular weight between the members of a first pair of compounds from the four compounds indicating which pair of components was incorporated into the pair of compounds in the second synthesis cycle, the difference in molecular weight between the members of a second pair of compounds from the four compounds indicating which pair of components was incorporated into the pair of compounds in the third synthesis cycle, the labeling indicating which component was added in the first synthesis cycle, and the total molecular weight of each compound, and the identity of the components added during the firs, second and third synthesis cycles, indicating which component was added during the fourth synthesis cycle.

57. A method of screening a library of compounds, comprising:
(a) in a first synthesis cycle, apportioning a plurality of supports into a plurality of first reaction vessels and reacting the supports with different first components in the different vessels, the first components attaching to the supports;
(b) in a second synthesis cycle,
(i) splitting the supports from each of the plurality of first reaction vessels into a set of multiple reaction vessels, the sets forming a plurality of second reaction vessels;
(ii) labeling the supports in each of the second reaction vessels with a different label, such that supports in a reaction vessel have the same label, but supports in different reaction vessels have different labels; and
(iii) reacting the supports in different reaction vessels of each set with different second components, whereby the second component attaches to the support via the first component;
(c) in a third synthesis cycle, pooling the supports from the plurality of second reaction vessels and reacting the supports with different third components in the different vessels, whereby the third components attach to the supports via the components added in a previous step;
(d) in a fourth synthesis cycle, pooling the supports, and apportioning the supports in a plurality of fourth reaction vessels; apportioning the supports in a plurality of third reaction vessels; and reacting the supports with different paired components, the members of each pair having a known difference in molecular weight, the difference in molecular weight differing between pairs, whereby the members of each pair attach independently to the support via a component added in a preceding step;
(e) in a fifth synthesis cycle, pooling the supports and apportioning the supports in a plurality of fifth reaction vessels, and reacting supports with different components, whereby the components attach to the support via a component added in the preceding step;
thereby forming a population of supports bearing different pairs of compounds, the members of the pairs of compounds having a known difference in molecular weight;
(f) separately assaying the supports bearing the paired compounds from each of the fifth plurality of reaction vessels, and isolating at least one support wherein at least one of the paired compounds on the isolated support has a desired property; and
(g) determining the molecular weights of each of the paired compounds borne by the at least one isolated support, the difference in molecular weight between the pair of compounds indicating which pair of components was incorporated into the pair of compounds in the fourth synthesis cycle, the labeling indicating which compounds were added in the first and second synthesis cycles, the fifth reaction vessel from which the support was obtained for the assaying step indicating which component was added during the fifth synthesis cycle, and the total molecular weight of each compound, and the identity of the components added during the first, second, fourth and fifth synthesis cycles, indicating which component was added during the third synthesis cycle.

58. The method of claim 57, wherein step (iii) is performed before step (ii).

59. The method of claim 57, wherein step (c) is performed before step (d).

60. The method of claim 57, wherein the third components each have different molecular weights.

61. A method for synthesizing a combinatorial library, comprising:
conducting a plurality of synthesis cycles to synthesize compounds on supports in a component-by-component fashion, a synthesis cycle comprising apportioning supports into reaction vessels and reacting the supports in different vessels with different components of the compounds, whereby the components attach to the supports or with components attached to the supports in previous steps, and the supports from different vessels are pooled between synthesis cycles;
wherein at least one cycle is conducted by contacting different vessels of supports with different first paired components, the members of each first pair attaching independently to the supports or components attached thereto in a previous cycle, whereby supports in the same vessel receive the same pair of components, and supports in different vessels receive different pairs of components, the components in each first pair having a known difference in molecular weight, and the differences in molecular weights varying between pairs, to produce a population of supports bearing different pairs of compounds, the members of the pairs of compounds having a known difference in molecular weight.

62. A method for encoding the identity of a component of a compound, comprising conducting a plurality of synthesis cycles, each cycle comprising reacting a support with a component of the compound, whereby the components attach to the support directly or via a component added in a previous cycle, wherein at least one cycle is conducted by contacting the support with a pair of components, each member of the pair attaching independently to the support or components attached thereto in a previous cycle, the components in each pair having a known difference in molecular weight which encodes for the identity of the pair of components.

63. A method for determining the identity of a component of a pair of compounds, comprising
(a) generating a correspondence regime in which pairs of components used to synthesize the pairs of compounds are encoded by the difference in molecular weight between the paired components, each pair of components having a different molecular weight difference;
(b) providing a support comprising a first and second compound, each comprising one component from a pair of components encoded by the correspondence regime; and
(c) determining the molecular weight difference between the first and second compound, the identity of a component of the compounds determined from the difference in molecular weight and the correspondence regime.

64. A support, comprising a first and second compound of differing composition comprising n components joined to one another via chemical bonds and differing in molecular weight, the difference in molecular weight encoding for a component of the first and second compound, and wherein the nth component is the same for the first and second compound.

65. The support of claim 64, wherein n=3, 4 or 5.

66. The support of claim 64, wherein the first component of the first and second compound is the same.

67. The support of claim 64, wherein the support comprises a label.

68. The support of claim 63, wherein the support further comprises a third and fourth compound that differ in composition and comprise n components joined to one another by chemical bonds and differing in molecular weight, the difference in molecular weight encoding for a component of the first, second, third and fourth compound, and wherein the nth component is the same for the first, second, third and fourth compounds.
